(19) United States
(12) Patent Application Publication
(10) Pub. No.: US 2018/0303075 A1
(43) Pub. Date: Oct. 25, 2018

(54) A MOUSE MODEL OF NONALCOHOLIC STEATOHEPATITIS AND USES THEREOF

(71) Applicant: VIRGINIA COMMONWEALTH UNIVERSITY, Richmond, VA (US)

(72) Inventors: Arun Sanyal, Mechanicsville, VA (US); Faridoddin Mirshahi, Richmond, VA (US)

(21) Appl. No.: 15/767,404

(22) PCT Filed: Oct. 12, 2016
(86) PCT No.: PCT/US16/56506

§ 371 Date: Apr. 11, 2018

Related U.S. Application Data
(60) Provisional application No. 62/240,223, filed on Oct. 12, 2015.

(51) Int. Cl. A01K 67/027

(52) U.S. Cl. CPC A01K 67/027; A01K 2267/0362; A01K 2227/105; A01K 2207/25

(57) ABSTRACT

An isogenic murine animal model for liver disease resulting from a Western (high fat, high sugar) diet is provided. This phenotypically and genotypically stable model sequentially develops steatosis (4-8 weeks), steatohepatitis (12-16 weeks), progressive fibrosis (16 week onwards) and spontaneous HCC (32-52 weeks), but only when fed a diet high in fat and sugar. The mice also develop obesity, insulin resistance and dyslipidemia, and the mouse hepatic transcriptome is concordant with the human NASH transcriptome at early and late time points, including activation of lipogenic, inflammatory and apoptotic signaling. The mouse HCC gene signature resembles S1 and S2 human HCC subclasses. This simple model of NASH and HCC that mirrors the development of human disease in terms of its triggers, serology, phenotype, histology, transcriptome and outcomes has utility in new target discovery, biomarker discovery, diagnostic test development, and screening and development of drugs for the corresponding liver conditions.

### CROSS-REFERENCE TO RELATED APPLICATIONS

This application claims priority to and the benefit of the filing date of U.S. Provisional Application No. 62/240,223 filed Oct. 12, 2015, the disclosure of which is hereby incorporated by reference herein in its entirety.

### FIELD OF THE INVENTION

This disclosure relates to the development of a genetically unique organism, namely an isogenic mouse strain that develops liver steatosis, fibrosis, steatohepatitis, cirrhosis and hepatocellular carcinoma when fed a Western diet.

### SEQUENCE LISTING

This document incorporates by reference a sequence listing text file submitted with this application on CD-ROMs in ASCII text format. The text file is named 02941029TAseqlistingfinal_ST25_rev.txt, is 3.45 gigabytes, and was created on Sep. 26, 2016.

### BACKGROUND OF THE INVENTION

Nonalcoholic fatty liver disease (NAFLD) has emerged as the leading cause of chronic liver disease in North America and most parts of the Western world¹. It has two principal phenotypes i.e. a fatty liver and steatohepatitis². Nonalcoholic steatohepatitis (NASH) is the more aggressive form of the disease and is expected to surpass hepatitis C virus infection as the leading etiology for hepatocellular cancer (HCC) and end-stage liver disease requiring liver transplantation³. Of great concern, a large proportion of NASH-related HCC occur in the absence of cirrhosis, the traditional risk factor for HCC^{4,5}. The growing prevalence of obesity and NASH underlies the rising incidence of HCC6. There is currently no approved therapy for NASH. Also, the initial promise of highly effective chemopreventive and therapeutic agents for HCC has not been realized.

The lack of a preclinical model for NASH that recapitulates the human disease is a barrier to therapeutic development. While a large number of models have been described⁷⁻¹³, their utility for preclinical identification of treatment targets and response to various interventions for drug development is limited. This is partly related to the diversity of molecular mechanisms that can induce fat accumulation in hepatocytes. Also, most models do not develop classical steatohepatitis that resembles the human disease⁷⁻¹³. Most models also do not develop progressive fibrosis. Some models are also not associated with insulin resistance, e.g. the methionine and choline deficient (MCD) diet⁷, while others require ablation of pancreatic beta cells^{10,11}. Besides not developing typical liver lesions, the substantially different hepatobiliary physiology of rats render rat-based models suspect. While the Ossabaw pig model comes close to mimicking human disease¹³, this is an expensive model and does not lend itself easily to gene manipulation to study the role of specific genes in disease pathogenesis. This lack of a viable model to test drugs in a preclinical setting before launching into expensive human trials is a deterrent for therapeutic development for NASH.

Similarly, there is a paucity of animal models for HCC that closely resemble human HCC. Most models require genetic manipulation or use of specific carcinogens and are thus at variance from the human condition¹⁴. As with NASH, the shortage of animal models for HCC remains a barrier for development of preventive and therapeutic strategies.

An ideal preclinical model for NASH and NASH-related HCC should be relatively simple, triggered by the same causes as human disease (caloric excess), associated with the same risk factors as in humans (obesity, insulin-resistance and dyslipidemia), and it should match human disease with respect to metabolic features, histology, outcomes, gene expression signature, lipid accumulation and activation of pathways relevant in humans. Importantly, it should also recapitulate the various stages of human disease. The development of HCC should also be triggered by the disease state and not by administration of a chemical carcinogen. Thus, a diet-induced animal model of nonalcoholic fatty liver disease was designed and invented to address this need for a better research tool that met all of the above criteria.

### SUMMARY OF THE INVENTION

The invention provides a murine animal model in which the development of NASH, NASH-related HCC, and other related disorders, is successfully mimicked. Significantly, the development of disease in the mouse does not rely on administering a chemical agent, but rather on providing the animal with a “Western style” diet that is high in fat and/or sugars. The mouse develops obesity, insulin-resistance and dyslipidemia and is a good match for human disease with respect to metabolic features, histology, outcomes, gene expression signature, lipid accumulation and disease activation of pathways related to disease development. Significantly, the mouse reproduces normally and is isogenic, i.e. the offspring consistently and reproducibly exhibit the same phenotype and disease profiles as the parents.

Accordingly, provided herein is a viable and fertile mouse, and offspring thereof, whose genome comprises any or all of the chromosome sequences represented by SEQ ID NOs:1-39. In some embodiments, administration of a high-fat and high-sugar diet causes development of one or more disease conditions selected from the group consisting of steatosis, fibrosis, steatohepatitis, cirrhosis, hepatocellular carcinoma, obesity, insulin resistance, and dyslipidemia.

Another aspect of the invention provides a method of inducing development of hepatic steatosis, progressive hepatic fibrosis, steatohepatitis, hepatocellular carcinoma, or a disorder associated with metabolic syndrome in a mouse whose genome comprises any or all of the chromosome sequences represented by SEQ ID NOs:1-39 comprising the steps of:

a. administering to said mouse a diet comprising standard mouse chow and water for at least the first 8 weeks after birth,

b. discontinuing administration of the diet comprising standard mouse chow and water when the mouse is at least 8 weeks old, and,

c. further administering to the at least 8 week old mouse a diet comprising high-fat mouse chow and sugar water for a duration of at least 4 weeks.

In some embodiments, the diet administered in step (c) is administered for at least 4 weeks and hepatic steatosis is induced. In other embodiments, the diet administered in step (c) is administered for at least 16 weeks and progressive hepatic fibrosis is induced. In other embodiments, the diet administered in step (c) is administered for at least 12 weeks and steatohepatitis is induced. In other embodiments, the diet administered in step (c) is administered for at least 32 weeks and hepatocellular carcinoma is induced. In other embodiments, the diet administered in step (c) is administered for at least 4 weeks and at least one disorder associated with metabolic syndrome is induced.

The metabolic syndrome that is induced in the methods of the invention includes, but is not limited to, impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, cardiovascular disease, atherosclerosis, microvascular disease, and kidney disease.

Another aspect of the invention provides a method for screening a compound for the prevention or treatment of a disorder selected from the group consisting of hepatic steatosis, progressive hepatic fibrosis, steatohepatitis, hepatocellular carcinoma, and a disorder associated with metabolic syndrome in a mouse whose genome comprises any or all of the chromosome sequences represented by SEQ ID NOs:1-39 comprising the steps of:

a. administering to said mouse a diet comprising standard mouse chow and water for at least the first 8 weeks after birth,

b. discontinuing administration of the diet comprising standard mouse chow and water when the mouse is at least 8 weeks old,

c. further administering to the at least 8 week old mouse a diet comprising high-fat mouse chow and sugar water for a duration of at least 4 weeks,

d. administering said compound concurrent with step (c) or after step (c), and

e. determining the effect of the compound on said disorder relative to a mouse not treated with the compound.

In some embodiments, the diet administered in step (c) is administered for at least 4 weeks and hepatic steatosis is induced. In other embodiments, the diet administered in step (c) is administered for at least 16 weeks and progressive hepatic fibrosis is induced. In other embodiments, the diet administered in step (c) is administered for at least 12 weeks and steatohepatitis is induced. In other embodiments, the diet administered in step (c) is administered for at least 32 weeks and hepatocellular carcinoma is induced. In other embodiments, the diet administered in step (c) is administered for at least 4 weeks and a disorder associated with metabolic syndrome is induced.

In some embodiments, the disorder associated with metabolic syndrome that is induced includes, but is not limited to, impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, cardiovascular disease, atherosclerosis, microvascular disease, and kidney disease.

Another aspect of the invention provides an in vivo model system for at least one condition selected from the group consisting of hepatic steatosis, progressive hepatic fibrosis, steatohepatitis, hepatocellular carcinoma, and a condition associated with metabolic syndrome comprising a viable and fertile mouse whose genome comprises any or all of the chromosome sequences represented by SEQ ID NOs:1-39, wherein said at least one condition is induced by administering to said mouse a diet comprising standard mouse chow and water for at least the first 8 weeks after birth, discontinuing administration of the diet comprising standard mouse chow and water when the mouse is at least 8 weeks old, and further administering to the at least 8 week old mouse a diet comprising high-fat mouse chow and sugar water for a duration of at least 4 weeks.

A further aspect of the invention provides a use of an in vivo model system according to claim 17 for: the study of at least one condition selected from the group consisting of hepatic steatosis, progressive hepatic fibrosis, steatohepatitis, hepatocellular carcinoma, and a condition associated with metabolic syndrome, or in vivo screening or testing of the efficacy of candidate drugs for the treatment of a condition recited in a).

### BRIEF DESCRIPTION OF THE DRAWINGS

FIG. 1A-H. Mice fed a high fructose/glucose, high fat Western Diet (WD SW) develop obesity, liver injury, dyslipidemia and insulin resistance. (a) Body weight change from mice fed a chow diet (CD NW) or high fructose/glucose, high fat Western Diet (WD SW) across the 52 week-time period. (b) Liver weight, (c) serum ALT and AST levels, (d) serum cholesterol, LDL-c and triglycerides levels, (e,f) insulin tolerance test (ITT) and (g,h) glucose tolerance test (GTT) were assessed on CD NW or WD SW mice at 8 or 52 weeks of diet. Data are expressed as the mean±SEM for 6-10 mice per group; *P<0.05 and **P<0.001, WD SW (top line) compared to CD NW (bottom line).

FIG. 2A-C. Mice fed a high fructose/glucose, high fat Western Diet (WD SW) sequentially develop a fatty liver, steatohepatitis, advanced fibrosis and liver tumors. (A) Gross liver from mice fed a chow diet (CD NW) or high fructose/glucose, high fat Western Diet (WD SW) for 8 (a-b), 16 (c-d) and 52 weeks (e-f). In mice fed a high fat Western Diet for 52 weeks, multiple foci of tumors were observed at the time of necropsy (h j) as compared to CD NW mice (g); areas of hemorrhage were seen in larger tumors (I). (B.C) Microscopic views of livers from CD NW or WD SW mice at 8 (a-d), 16 (e-h) or 52 weeks (i-l) of diet. Representative liver sections stained with hematoxylin-eosin (H&E) (B) or picosirius (C) are shown. Original magnification, ×5 (a-b, e-f, i-j) and ×20 (c-d, g-h, k-l). WD SW mice liver display both macrovesicular and small droplet steatosis at 8 weeks (b,d). At 16 weeks, WD SW mice develop steatohepatitis with predominantly macrovesicular steatosis, hepatocelullar ballooning, foci of inflammation and well-established perisinusoidal fibrosis (f.h). At 52 weeks, aggressive steatohepatitis is present in WD SW mice with extensive bridging fibrosis with early nodule formation (j,l).

FIG. 3A-B. Mice fed a high fructose/glucose, high fat Western Diet (WD SW) develop the most florid macrovesicular steatosis, lobular inflammation and hepatocellular ballooning, apoptotic bodies and occasional Mallory-Denk bodies. (A) Representative images of hematoxylin-eosin (H&E) staining of liver tissue from mice fed a high fructose/glucose, high fat Western Diet (WD SW) for 52 weeks depicting the individual component of steatohepatitis (as indicated by arrow): (a) hepatocyte ballooning, (b) Mallory-Denk bodies, (c) lobular inflammation and (d) apoptotic bodies. Original magnification, ×40 (B) Histology score for steatosis, hepatocyte ballooning, lobular inflammation, NAFLD Activity Score, fibrosis and Collagen Proportional Area (CPA) were quantified on histologic liver sections from mice fed for 8 or 52 weeks either a chow diet (CD NW), a high fructose/glucose diet (CD SW), a high fat Western Diet (WD NW) or a high fructose/glucose, high fat Western Diet (WD SW). Data are expressed as the mean±SEM for 6-10 mice per group; *P<0.05, CD SW, WD NW or WD SW compared to CD NW.

FIG. 4A-F. Activation of signaling pathways relevant in human NASH in the liver of mice fed a high fructose/glucose, high fat Western Diet (WD SW). (A) Whole cell lysates were prepared from liver tissue from mice fed a chow diet (CD NW) or high fructose/glucose, high fat Western Diet (WD SW) for 8 or 52 weeks. Immunoblot analysis were performed for Fatty Acid Synthase (FAS)-N, phosphorylated and total Acetyl-CoA Carboxylase (p-ACC and t-ACC), phosphorylated and total JNK (p-JNK and t-JNK), phosphorylated and total p42/p44 (p-p42/p44 and t-p42/p44), PUMA, BIM and caspase-3 (C3) displaying cleaved caspase-3 product p18. The cleaved form of C3 was only visualized after long exposure times. β-actin was used as a control for protein loading. Bands were cut and combined from the same radiograph. (B-F) Transcriptome analysis using the Illumina mouse WG6 Expression BeadChip kits (Illumina) was performed on liver tissues from CD NW or WD SW mice after 8 weeks of diet (n=5 per group). The data are presented as: (B) volcano plot of changes in gene expression levels and genes that are significantly up- or down-regulated; (C) Heatmap demonstrating deregulated genes in WD SW versus CD NW mice. High and low gene expression are indicated. (D) Gene ontology (GO) processes; (E) Gene set Enrichment Analysis (GSEA); and (F) Process Networks analysis. The top rank ordered processes, maps and networks are based on statistical significance.

FIG. 5A-H. Hepatic gene expression dataset in mouse fed a high fructose/glucose, high fat Western Diet (WD SW) for 52 weeks concords with a human liver cirrhosis and NASH-associated gene signature. Transcriptome analysis using the Illumina mouse WG6 Expression BeadChip kits (Illumina) was performed on liver tissues from CD NW or WD SW mice after 52 weeks (A-H) and 8 weeks (H) of diet (n=5 per group). (A) Gene ontology (GO) processes; (B) Gene set Enrichment Analysis (GSEA); (C) Process Networks analysis. The top rank ordered processes, maps and networks are based on statistical significance. (D) Volcano plot of changes in gene expression levels and genes that are significantly up- or down-regulated are indicated; (E) Heatmap demonstrating deregulated genes in WD SW versus CD NW mice. High and low gene expression are indicated. (F-G) A 186-gene signature (73 poor prognosis-correlated (F) and 113 good prognosis-correlated (G) genes) prognostic in liver cirrhosis and HCC patients with mixed etiologies was strikingly induced in the livers of WD SW 52 weeks mice by GSEA. NES, normalized enrichment score; FDR, false discovery rate. (H) Similarity of global liver transcriptome of mice fed a high fructose/glucose, high fat Western diet (WD SW) for 8 or 52 weeks with human NASH. Global liver transcriptome in liver biopsy tissues from 18 human NASH patients and 41 normal/healthy obese individuals was compared to WD SW mice using Subclass Mapping algorithm. Striking similarity between WD SW-fed mice and human NASH was observed for 8 weeks and 52 weeks. Numbers on the heatmaps indicate FDR values for the transcriptome similarity.

FIG. 6A-G. Tumors gene signature in mice fed a high fructose/glucose, high fat Western Diet (WD SW) for 52 weeks. (A) Microscopic views of adenomas (a,b,c) and hepatocarcinomas (HCC) tumors (d,e,f) from WD SW mice at 52 weeks of diet. (a) adenoma, (hematoxylin-eosin (H&E), original magnification, ×2.5); (b cords of hepatocytes with mild atypia and trabecular organization (H&E, original magnification, ×20); (c) unpaired artery between hepatocytes with mild anisocaryosis (H&E, original magnification, ×40); (d) a basophilic well-demarcated tumor with a satellite nodule and with steatosis in the background liver (H&E, original magnification, ×2); (e) interface between malignant tumor (top half) and non-tumoral liver. The lobules of tumoral cells show marked anisocaryosis, eosinophilic cytoplasm, irregular basophilic nuclei and loss of sinusoidal architecture (H&E, original magnification, ×40); (f) satellite nodules made of clusters of tumoral cells (arrow) and dysplastic foci with multinucleated irregular hepatocytes (arrow) (H&E, original magnification, ×20). (B-G) Transcriptome analysis using the Illumine mouse WG6 Expression BeadChip kits (Illumina) was performed on liver adjacent to tumors (WD SW) or tumor tissue (HCC) from WD SW mice after 52 weeks of diet (n=5 per group). (B) Gene signatures of human HCC subclasses S1, S2, and S3 were analyzed in WD SW-induced HCCs in comparison to non-tumor livers from WD SW mice at 52 weeks. Gene Set Enrichment Analysis (GSEA) revealed significant induction of S1 and S2 subclass signatures in WD SW-induced HCCs. The induction was relatively stronger for S1 subclass which was associated with steatohepatitic HCC variant. (C) Volcano plot of changes in gene expression levels and genes that are significantly up- or down-regulated are indicated; (D) Heatmap demonstrating deregulated genes in WD SW-induced tumors (Tumors) versus tissues adjacent to tumors from WD SW mice at 52 weeks (WD SW). high and low gene expression are indicated; (E) Gene ontology (GO) processes; (F) Gene set Enrichment Analysis (GSEA); and (G) Process Networks analysis. The top rank ordered processes, maps and networks are based on statistical significance.

FIG. 7A-B. WD SW mice develop steatosis. (A,B) Representative Oil O-stained liver cryostat sections from livers from WD SW mice at 52 weeks of diet. Original magnification, ×10.

FIG. 8A-B. Florid steatohepatitis induced by a high fructose/glucose, high fat Western diet (WD SW) for 52 weeks presented with macrovesicular steatosis, lobular inflammation and hepatocellular ballooning and occasional Mallory-Denk bodies. Additional representative images of hematoxylin-eosin (H&E) staining of liver tissue from mice fed a high fructose/glucose, high fat Western diet (WD SW) for 52 weeks depicting (A) extensive macrovesicular steatosis, focal hepatocellular ballooning (arrows) and lobular inflammation (arrows) and (B) Mallory-Denk bodies were noted (arrows). Original magnification, ×20.

FIG. 9. Florid steatohepatitis and fibrosis develops in mice fed a high fructose/glucose, high fat Western Diet (WD SW) for 16 weeks. Histology score for steatosis, hepatocyte ballooning, lobular inflammation, NAFLD Activity Score, and fibrosis were quantified on histologic liver sections from mice fed for 16 weeks either a chow diet (CD NW), a high fructose/glucose diet (CD SW), a high fat Western Diet (WD NW) or a high fructose/glucose, high fat Western Diet (WD SW). Data are expressed as the mean±SEM for 5-9 mice per group; *P<0.05, WD SW compared to CD NW.

FIG. 10A-B. Only the combination of high fructose/glucose diet with a high fat Western diet (WD SW) sequentially induce a NASH phenotype with macrovesicular steatosis and advanced fibrosis. (A-B) Microscopic views of livers from mice fed for 8 or 52 weeks either a chow diet (CD NW) (a,e,i,m), a high fat Western diet (WD NW) (b,f,j,n), a high fructose/glucose diet (CD SW) (c,g,k,o) or a high fructose/glucose, high fat Western diet (WD SW) (d,h,l,p). Representative liver sections stained with hematoxylin-eosin (A) or picosirius (B) are shown. Original magnification, ×5 (a-d, i-l) and ×20 (e-h, m-p). WD SW mice develop the most florid steatohepatitis and progressive fibrosis.

FIG. 11A-C. Top rank Gene Set Enrichment Analysis based on statistical significance in mice fed a high fructose/glucose, high fat Western diet (WD SW) for 8 weeks. (A) Blood coagulation pathway. (B) Cytoskeleton remodeling-TGF,WNT and cytoskeletal remodeling. (C) LRRK2 in neurons in Parkinson's disease. Pathways maps were visualized using the MetaCore pathway analysis suite (Thomson Reuters, New York, N.Y.). Experimental data from all files is linked to and visualized on the maps as thermometer like figures. Up ward thermometers indicate up regulated signals and down ward ones indicate down regulated expression levels of the genes.

FIG. 12A-B. Hepatic adenoma in mice fed a high fructose/glucose, high fat Western diet (WD SW) for 52 weeks. (A-B) Additional microscopic views of livers from WD SW mice at 52 weeks of diet. Liver sections stained with hematoxylin-eosin (A) or trichrome (B) depicting a well-delineated benign hepatic adenoma. Original magnification, ×2.5.

FIG. 13A-C. Top rank Gene Set Enrichment Analysis based on statistical significance in mice fed a high fructose/glucose, high fat Western diet (WD SW) for 52 weeks. (A) Androstenedione and testosterone biosynthesis and metabolism p.1/rodent version. (B) Role of ZNF202 in regulation of expression of genes involved in atherosclerosis. (C) Oxidative stress_Role of IL-8 signaling pathway in respiratory burst. Pathways maps were visualized using the METACORE™ pathway analysis suite (Thomson Reuters, New York, N.Y.). Experimental data from all files is linked to and visualized on the maps as thermometer like figures. Upward thermometers indicate up-regulated signals and down ward ones indicate down-regulated expression levels of the genes.

### DETAILED DESCRIPTION

Described herein is a novel murine animal model with utility for the study of the development and treatment of disease conditions resulting from Western diet, and particularly liver disorders arising in humans. This new Diet-Induced Animal Model of Nonalcoholic fatty liver Disease (DIAMOND™) mouse model comprises an isogenic strain derived from a cross of two common mouse strains, 129S1/SvImJ and C57BI/6J where a simple high fat diet accompanied by ad lib consumption of water with a high fructose and glucose content (Western Diet sugar water (WD SW)) sequentially induces steatosis, steatohepatitis, progressive fibrosis and HCC. The mouse genome has the genetic sequence comprising any or all of the chromosome sequences represented by SEQ ID NOs:1-39. In exemplary embodiments, the mouse has a genetic sequence that comprises or consists of a sequence at least 90, 91, 92, 93, 94, 95, 96, 97, 98, 99 or 100% identical to any or all of the chromosome sequences represented by SEQ ID NOs:1-39.

SEQ ID NOs 1 and 2 together comprise a representative sequence of chromosome 10. SEQ ID NOs 3 and 4 together comprise a representative sequence of chromosome 11. SEQ ID NOs 5 and 6 together comprise a representative sequence of chromosome 12. SEQ ID NOs 7 and 8 together comprise a representative sequence of chromosome 13. SEQ ID NOs 9 and 10 together comprise a representative sequence of chromosome 14. SEQ ID NOs 11 and 12 together comprise a representative sequence of chromosome 15. SEQ ID NO: 13 comprises a representative sequence of chromosome 16. SEQ ID NO: 14 comprises a representative sequence of chromosome 17. SEQ ID NO: 15 comprises a representative sequence of chromosome 18. SEQ ID NO: 16 comprises a representative sequence of chromosome 19. SEQ ID NOs 17, 18, and 19 together comprise a representative sequence of chromosome 1. SEQ ID NOs 20 and 21 together comprise a representative sequence of chromosome 2. SEQ ID NOs 22 and 23 together comprise a representative sequence of chromosome 3. SEQ ID NOs 24 and 25 together comprise a representative sequence of chromosome 4. SEQ ID NOs 26 and 27 together comprise a representative sequence of chromosome 5. SEQ ID NOs 28 and 29 together comprise a representative sequence of chromosome 6. SEQ ID NOs 30 and 31 together comprise a representative sequence of chromosome 7. SEQ ID NOs 32 and 33 together comprise a representative sequence of chromosome 8. SEQ ID NOs 34 and 35 together comprise a representative sequence of chromosome 9. SEQ ID NO: 36 comprises a representative sequence of chromosome M. SEQ ID NOs 37 and 38 together comprise a representative sequence of chromosome X. SEQ ID NO: 39 comprises a representative sequence of chromosome Y.

The inventors have deposited a hepatocyte cell culture having the genomic DNA sequence of a mouse as described herein at the American Type Culture Collection (ATCC, 10801 University Boulevard, Manassas, Va. 20110), in accordance with the terms of Budapest Treaty on Oct. 11, 2016. The deposited cell culture has the ATCC deposit number PTA-123551.

The inventors observed that, in the original non-isogenic cross-bred mice, 12951/SvImJ:C57BI/6J cross mice (termed 129/B6), a small percentage developed insulin resistance, obesity and liver pathology similar to humans when fed a Western diet. However, these heterogenous 129/B6 progeny do not have utility as a model of human disease because they are not isogenic and all of the mice do not consistently develop the pathology in response to the trigger (being fed a Western diet). Therefore, the inventors selectively bred the progeny by crossing mice that did develop the disease conditions to one another. After many generations were crossed over a period of years, a strain was obtained in which the histological phenotype of steatohepatitis was obtained consistently when the mice were fed the trigger diet. The mice were confirmed to be isogenic and the genetic sequence of the genome was determined (SEQ ID NOs:1-39).

In some aspects, the mice of the invention are fed a high fat, sugar water diet in order to induce one or more diseases or conditions of interest. As used herein, “ ”high fat” refers to food (e.g. rat chow) with a fat content of at least 42 energy % and 0.2% cholesterol. The chow diet typically contains about 5.8% fat. The Western diet may contain about 12.8% saturated fat. The presence of saturated FFA and cholesterol is important for disease progression into steatohepatitis. As used herein, “sugar water” refers to an aqueous solution comprising at least about 23.1 g/L d-fructose+18.9 g/L d-glucose of a “sugar” or sugar source such as glucose, fructose, sucrose, high fructose corn syrup.

The DIAMOND™ mouse has great utility as a research tool. The pattern of gene and protein expression can be studied during development and progression of the diseases from steatosis, to steatohepatitis, progressive fibrosis, cirrhosis, and hepatocellular carcinoma. Other comorbidities other than liver disease can be studied using this model, including various components of metabolic syndrome. Metabolic syndrome is recognized in the art as a constellation of associated disorders or conditions which are characteristic of metabolic syndrome. Non-limiting examples of disorders included in this group include impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, hypercholesterolemia, hypertriglyceridemia, cardiovascular disease, macrovascular disease, atherosclerosis, microvascular disease, and kidney disease.

Thus the model may be exploited to discover the metabolic pathways that are actively involved in the development of the constellation of diseases discussed above. Experiments can be designed using the new mouse model to discover treatments that interrupt the disease process and halt disease development, and/or to treat or reverse already developed diseases. The model is particularly useful as a screening tool for new compounds in pre-clinical drug development. Specifically, mice may be dosed with compounds or drugs at any point during the development of any of the disease conditions to determine if the compound or drug has therapeutic value. In particular, if symptoms of disease or other disease markers increase or stay the same after administration of the compound or drug as compared to a suitable control mouse (e.g. an isogenic mouse that is fed the same diet but does not receive the compound or drug), then the compound or drug is determined to be ineffective. Alternatively, if symptoms of disease or other disease markers decrease after administration of the compound or drug as compared to a suitable control mouse, then the compound or drug is determined to be therapeutically effective. Because the physiology and pathology of the DIAMOND™ mouse closely resembles the physiology and pathology of humans during disease development, compounds or drugs that prevent or treat disease in the mice are more likely to be effective in human clinical trials. Thus, use of the mice as a screening tool will speed up the pre-clinical drug development process and enable better decision-making regarding which compounds or drugs should advance to human clinical trials.

It will be appreciated that although the methods in this application are directed primarily towards dosing the DIAMOND™ mice with compounds or drugs to prevent or treat disease, these are non-limiting examples and the utility of the mice extends beyond pharmaceutical screening purposes. Additionally, while the dietary regime described herein comprises allowing the mice to “grow up” on a normal chow diet before being switched to a “Western” diet at around 8 weeks of age, this is not the only useful schedule. For instance, in some embodiments, the female breeders are not fed a Western diet. However, epigenetic changes in organisms may result due to diets of mothers before and during pregnancy, or in immature individuals. Therefore, in some embodiments, the mice begin the Western diet prior to 8 weeks, e.g. prior to sexual maturity. In some embodiments, the Western diet is fed to breeders of either sex for various durations prior to mating and/or during pregnancy. Such alterations in protocols yield useful information regarding epigenetics, fertility, etc.

Furthermore, in some embodiments, animals are dosed with compounds or drugs during administration of the Western diet as disease develops, or at timepoints after diseases have developed and progressed. However, it will be appreciated that compounds or drugs may be administered to the mice at any time, including prior to initiating the Western diet, for prevention studies. In some embodiments, the DIAMOND™ mouse is used as an experimental platform for creation of “knock-ins” and “knock-outs” to investigate the effects of adding and deleting genes.

Further, agents other than “drugs” may be tested using the mice, e.g. various natural products, so-called “nutraceuticals”, herbs, dietary additives such as probiotics, vitamins, etc. may be tested to determine their effects on disease occurrence and/or progression; as may changes (variations) in diet (e.g. varying the amount or type of fats and sugars, etc.); and/or activity levels (mimicking increased or decreased exercise), etc.

While the majority of the data obtained on the DIAMOND™ mouse physiology and pathology presented herein concerns liver pathology and some blood-borne proteins and metabolites, it is expressly noted that other organ systems and biological fluids may and do show abnormalities. Thus the utility of this mouse model extends beyond liver disease and blood-based biomarkers and into all areas of physiology known to be affected by the development of metabolic syndrome, including but not limited to the cardiovascular system, the muscular system, the endocrine system, the reproductive system, the digestive system, the kidneys, and the adipose tissue.

It has been postulated that the microbiome of the gut plays a role in the development of liver disease. Another useful aspect of this model, therefore, is the ability to inoculate the gut with various organisms and determine if the changes in the gut microbiome alter disease development and progression. Thus the model has utility for the discovery and development of pro-biotics and gut flora supplements. Accordingly, in some embodiments we contemplate the use of a “clean” DIAMOND™ mouse model, and in other embodiments we contemplate the addition of one or more commensal or pathogenic bacterial, viral, or fungal species to the model.

The mouse of the invention is also useful for biomarker discovery studies. For example, studies of circulating lipids, proteins and nuclear material could be used for biomarker development. Also, changes in the microbiome and measurement of microbial metabolites and linking them to disease phenotype would guide biomarker development efforts.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

All examples and descriptions in this application are non-limiting and shall not be construed as the only way that the invention can be practiced. Variations in methodology and protocol that are within the spirit of the invention are also claimed.

### Example

### The Genetic Background of the Mouse:

Two pure wholly genetically characterized mouse strains were cross-bred. These two parental strains are 129S1/SvImJ and C57BI/6J. The progeny that resulted from the cross-breeding are called 129/B6 mice. The first progeny were heterogeneous (not genetically identical to one another). In order to transform the heterogeneous progeny into an isogenic mouse strain in which all individuals were equally genetically susceptible to developing NAFLD, NASH and HCC in response to Western diet, further selective inbreeding was carried out for over 20 generations to yield the isogenic strain of DIAMOND™ mice that all develop disease pathology in response to dietary trigger. The isogenic status of the mice was confirmed by testing for a set of 158 single nucleotide polymorphisms (SNPs) that included both specific C57BI/6 and 129S1 SNPs in 6 randomly chosen mice from the colony (Table 1). This SNP testing demonstrated that approximately 60% of the genetic marker SNPs were of C571BI/6 origin while 40% of the genetic marker SNPs were of 129S1 origin. Importantly, all mice were genetically identical. The sequence of the entire genome of the new mouse was determined (SEQ ID NOs:1-39). The development of the new organism with unique genetic makeup and utility as a model of human disease was complete.

TABLE 1
Background strain characterization of the 129S1/SvlmJ;
C57BI/6J mouse strain

| Chromosome # | SNPs B6 | SNPs S129 |
|---|---|---|
| (# of SNPs analyzed) | background (%) | background (%) |
| Chromosome 1 (11 SNPs) | 27.3 | 72.7 |
| Chromosome 2 (10 SNPs) | 70.0 | 30.0 |
| Chromosome 3 (9 SNPs) | 22.2 | 77.8 |
| Chromosome 4 (9 SNPs) | 77.8 | 22.2 |
| Chromosome 5 (9 SNPs) | 100.0 | 0.0 |
| Chromosome 6 (9 SNPs) | 88.9 | 11.1 |
| Chromosome 7 (7 SNPs) | 57.1 | 42.9 |
| Chromosome 8 (7 SNPs) | 57.1 | 42.9 |
| Chromosome 9 (8 SNPs) | 87.5 | 12.5 |
| Chromosome 10 (8 SNPs) | 87.5 | 12.5 |
| Chromosome 11 (7 SNPs) | 57.1 | 42.9 |
| Chromosome 12 (8 SNPs) | 37.5 | 62.5 |
| Chromosome 13 (7 SNPs) | 28.6 | 71.4 |
| Chromosome 14 (7 SNPs) | 42.9 | 57.1 |
| Chromosome 15 (5 SNPs) | 60.0 | 40.0 |
| Chromosome 16 (6 SNPs) | 100.0 | 0.0 |
| Chromosome 17 (6 SNPs) | 50.0 | 50.0 |
| Chromosome 18 (7 SNPs) | 28.6 | 71.4 |
| Chromosome 19 (5 SNPs) | 100.0 | 0.0 |
| Chromosome X (12 SNPs) | 58.3 | 41.7 |
| Total (%) | 61.1 | 38.9 |

Analysis was performed on DNA isolated from six 129S1/SvlmJ; C57BI/6J strain mice;
SNPs complete background analysis was identical throughout the different mice.

### WD SW-Fed Mice Develop Obesity, Liver Injury and Dyslipidemia:

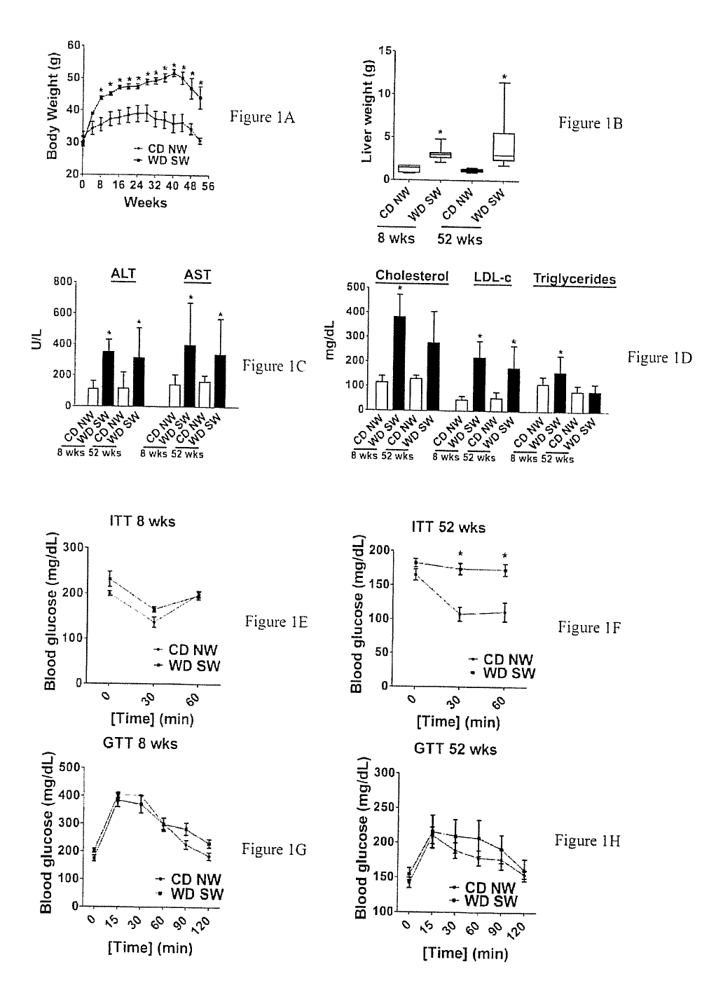

Chow diet normal water (CD NW)-fed mice gained a small amount of weight compared to baseline values but essentially had stable weights from 8 to 52 weeks (FIG. 1A and Table 2). In contrast, mice fed a WD SW became significantly heavier as compared to concurrent CD NW-fed controls (p<0.001). The weight gain peaked by 8 weeks with non-significant minor additional weight gain between 8 and 52 weeks following initiation of the diet. The liver weight was also significantly higher in mice fed WD SW compared to CD NW-fed controls at 8 weeks and subsequent time points including week 52 (FIG. 1B).

TABLE 2
Serum biochemical parameters

| | CD NW | CD SW | WD NW | WD SW |
|---|---|---|---|---|
| 8 weeks of diet | | | | |
| Body weight (g) | 32.4 ± 5.5 | 35.3 ± 4.4 | 35.2 ± 2.1 | 43.4 ± 1.9*** |
| AST (U/L) | 141.1 ± 64.9 | 133.1 ± 76.9 | 1238.7 ± 546.0 | 397.6 ± 273.5* |
| ALT (U/L) | 112.0 ± 52.0 | 65.7 ± 30.1 | 731.0 ± 575.7 | 350.8 ± 80*** |
| Alkaline Phosphatase | 60.6 ± 7.5 | 59.4 ± 12.5 | 61.5 ± 8.1 | 118.0 ± 38.8*** |
| (U/L) | | | | |
| Cholesterol (mg/dL) | 114.3 ± 26.4 | 118.9 ± 34.8 | 181.3 ± 26.9 | 380.4 ± 92.2*** |
| LDL-c (mg/dL) | 45.1 ± 14.4 | 44.6 ± 22.1 | 79.0 ± 16.8 | 218.0 ± 66.5*** |
| Triglycerides (mg/dL) | 111.4 ± 29.1 | 96.0 ± 20.9 | 108.7 ± 5.8 | 159.6 ± 67.4* |
| 52 weeks of diet | | | | |
| Body weight (g) | 30.9 ± 2.3 | 34.7 ± 3.9 | 50.5 ± 8.5*** | 44.2 ± 7.4** |
| AST (U/L) | 162.0 ± 38.8 | 161.0 ± 25.4 | 628.4 ± 236.3*** | 338.2 ± 230.1 |
| ALT (U/L) | 117.3 ± 71.4 | 104.0 ± 56.7 | 560.5 ± 360.7* | 314.2 ± 191.2 |
| Alkaline Phosphatase | 100.0 ± 45.1 | 102.0 ± 22.7 | 132.0 ± 73.6 | 155.1 ± 104.1 |
| (U/L) | | | | |
| Cholesterol (mg/dL) | 130.0 ± 13.3 | 100.0 ± 26.9 | 337.4 ± 113.6** | 276.4 ± 128.6 |
| LDL-c (mg/dL) | 53.3 ± 23.7 | 43.0 ± 11.5 | 209.8 ± 94.3** | 176.0 ± 91.1* |
| Triglycerides (mg/dL) | 81.3 ± 24.9 | 82.0 ± 18.0 | 109.6 ± 22.3 | 82.2 ± 30.4 |

Mice were fed for 8 or 52 weeks either a chow diet (CD NW), a high fructose/sucrose diet (CD SW), a high fat Western diet (WD NW) or a high fructose/sucrose, high fat Western diet (WD SW).
Values are mean ± SEM for 6-10 mice per group;
***P < 0.001,
**P < 0.01,
*P < 0.05 CD SW, WD NW or WD SW compared to CD NW.
ALT, alanine aminotransferase;
AST, aspartate aminotransferase;
LDL-c, low-density lipoprotein-cholesterol.

This was accompanied by an increase in aspartate aminotransferase (AST) and alanine aminotransferases (ALT) in WD SW-fed mice compared to CD NW-fed mice (p<0.05 for AST and <0.001 for ALT) (FIG. 1C). Mice fed a WD SW also developed an increase in total cholesterol and LDL-cholesterol (LDL-c), as it has been reported in humans with NASH15. Hypertriglyceridemia also developed in these mice and was maximal at 8 weeks (p<0.05) (FIG. 1D). These lipid changes were greatest within the first 8-16 weeks and started to decline after week 24 (data not shown). At week 52, LDL-c remained significantly elevated while the hypertriglyceridemia declined to values noted in CD NW-fed mice.

### WD SW-Fed Mice Develop Insulin Resistance:

Insulin resistance was measured by an insulin tolerance test (ITT) that was performed at an early time point after initiation of the diet at 8 weeks and then at 52 weeks. This was done to assess the initial metabolic response to the diet and to determine if these changes were sustained. At 8 weeks, following insulin administration, the blood glucose drop in mice receiving WD SW was less when compared to CD NW-fed controls (FIG. 1E). At week 52, WD SW-fed mice had significant insulin resistance (p<0.05) and an essentially flat glucose profile following insulin administration (FIG. 1F). CD NW-fed mice had a normal expected drop in glucose in response to insulin administration. On a separate day, a glucose tolerance test (GTT) was also performed using standard methods¹⁶. While glucose levels were often higher following intra-peritoneal glucose administration on both weeks 8 and 52 in mice fed WD SW (FIG. 1G-H), these differences did not reach statistical significance. This indicated that despite insulin resistance, the mice still had enough pancreatic islet β cell reserve to be able to mount a gluco-regulatory response after a glucose challenge.

### WD SW-Fed Mice Sequentially Develop a Fatty Liver, Steatohepatitis and Advanced Fibrosis:

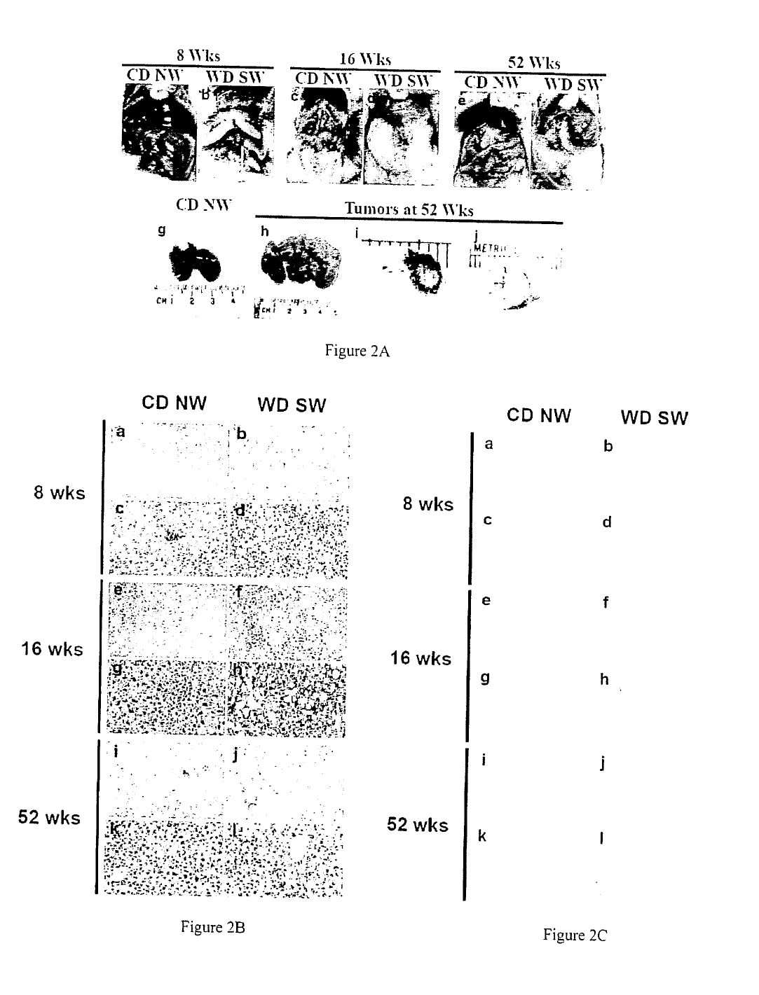

Gross morphology of the liver: Within 4-8 weeks of WD SW administration, the liver became tan and visibly lighter in color compared to CD NW-fed mice for the same duration (FIG. 2A). This was maintained up to 52 weeks. Between weeks 32-52 the surface of the liver also demonstrated nodularity in some but not in all mice. Also both single and multiple foci of tumors were seen at the time of necropsy. Some of the larger tumors had areas of hemorrhage in them. These findings are consistent with HCC that develops in humans with NASH.

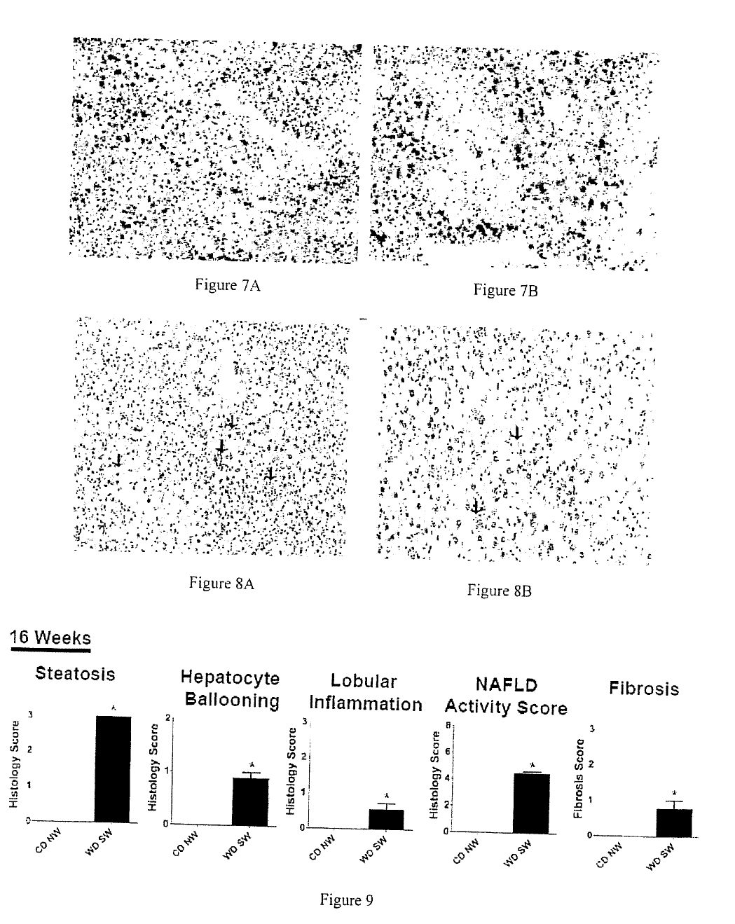

Development of steatosis, steatohepatitis and progressive fibrosis: CD NW-fed mice had normal hepatic architecture and histology at all time-points studied (FIG. 2B). They also had no excess of collagen in the liver as assessed by Sirius Red staining (FIG. 2C). In contrast, mice fed a WD SW developed extensive hepatic steatosis between weeks 4-8 (FIG. 2B). The steatosis included macrovesicular steatosis along with small droplet lipid accumulation. The latter was more commonly seen around central veins. The small droplet lipid changes decreased over time while the macrovesicular steatosis increased progressively by week 52. Steatosis was further confirmed by Oil-Red-O staining (FIG. 7A-B). Although there was minimal or no visible inflammation, about half the mice had evidence of some hepatocellular ballooning by week 8. There was also no significant increase in fibrosis by week 8 (FIG. 2C).

Between weeks 12-16, steatohepatitis developed in mice fed WD SW (FIG. 26). In addition to the continued presence of mainly macrovesicular steatosis, both inflammation and hepatocellular ballooning and occasional Mallory-Denk bodies were noted (FIG. 2B, 3A and FIG. 8A-B). There was also evidence of apoptotic bodies. With passage of time and continued WD SW administration, the ballooning and Mallory bodies were seen more frequently. At week 52, the inflammation and ballooning was seen in all mice studied. The inflammation was mainly lobular as seen in humans with steatohepatitis. Pericellular fibrosis was seen at week 16 and increased over time. By week 52, advanced bridging fibrosis and even early nodule formation could be seen (FIG. 2C).

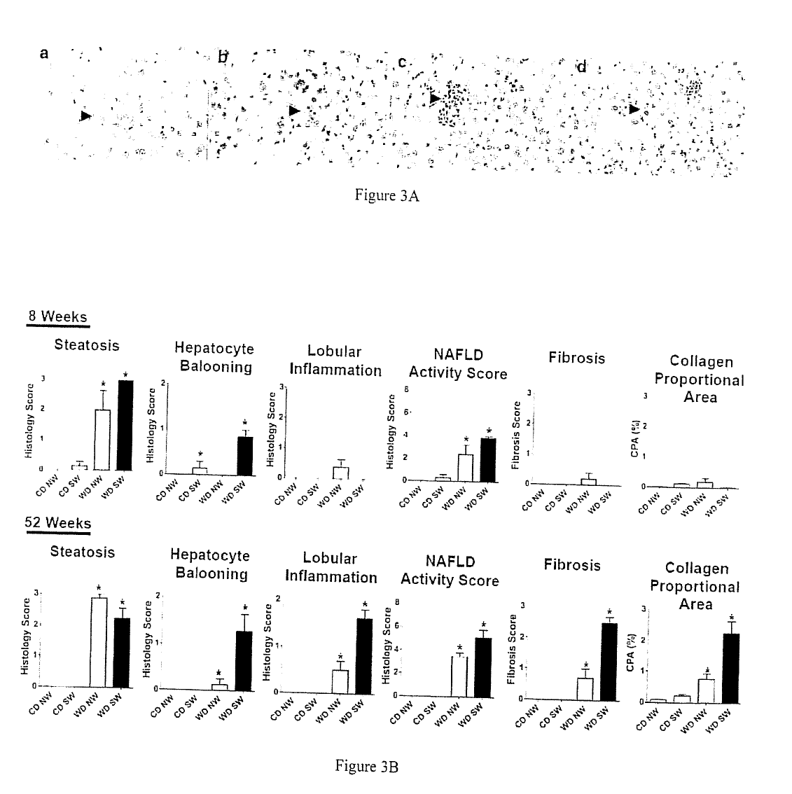

Having established the presence of steatohepatitis and the individual components of steatohepatitis seen in humans in mice fed WD SW, the severity of the individual lesions were quantified using the NASH Clinical Research Network (CRN) criteria and calculation of the NAFLD activity score (NAS)¹⁷. All mice fed WD SW developed grade 3 macrovesicular steatosis by week 8 (FIG. 3B). By week 52, the steatosis grade declined modestly but was still significantly greater than in CD NW-fed mice which did not develop any steatosis. The severity of hepatocyte ballooning at week 52 was higher compared to both CD NW-fed mice and mice fed WD SW for 8 or 16 weeks. Lobular inflammation developed by week 16 (FIG. 9) and all mice had substantial lobular inflammation even at week 52. As expected, these changes translated in to progressively increased NAS from week 8 to 52. However, at week 8, the increase was driven by steatosis while at week 52, steatosis, inflammation and ballooning all contributed to the increase in NAS.

To confirm the progressive increase in fibrosis over time after administration of WD SW, quantitative morphometry was performed on Sirius Red-stained liver sections (FIG. 3B). The collagen proportional area (CPA) was not significantly different from CD-fed controls at 8 weeks. There was however a progressive increase in the CPA from week 8 to 16 and then 52 in mice fed WD SW while CD fed mice did not demonstrate any fibrosis.

### Effects of Gender, Diet and Genotype on Disease Phenotype:

Female mice also developed steatosis at week 8 and steatohepatitis by week 16 (data not shown). They developed increasing fibrosis but did not develop fully established cirrhosis by week 52. The severity of the steatohepatitis was also milder than in male mice. To determine the contribution of high fat diet alone (Western Diet (WD NW)) and sugar water (CD SW) alone, independent experiments where the mice were fed WD NW or CD SW was performed.

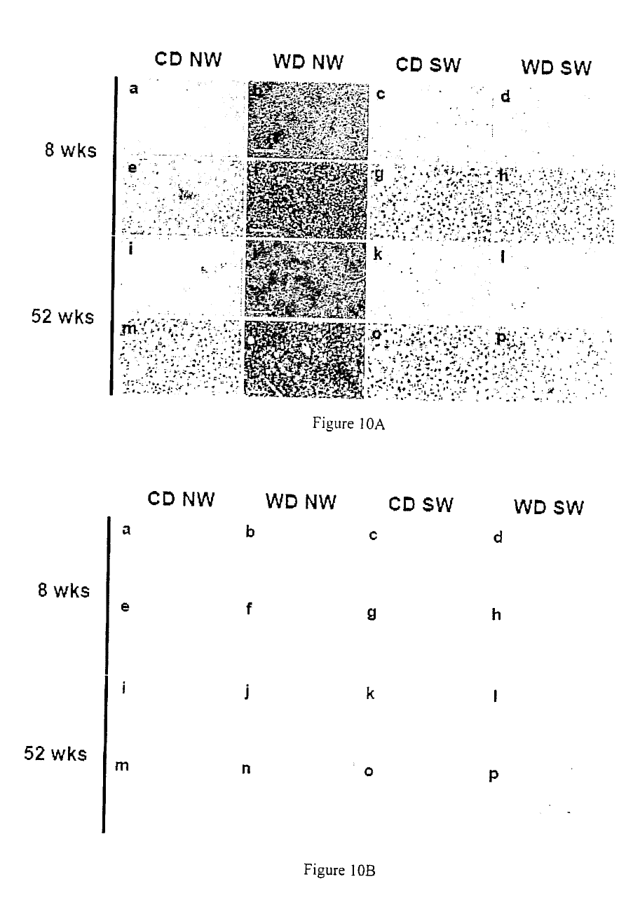

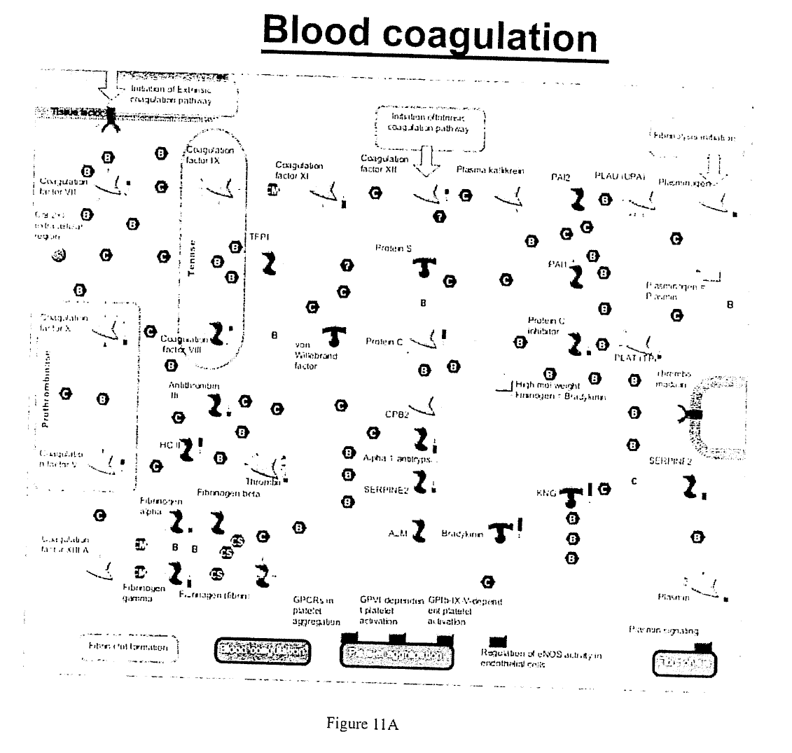

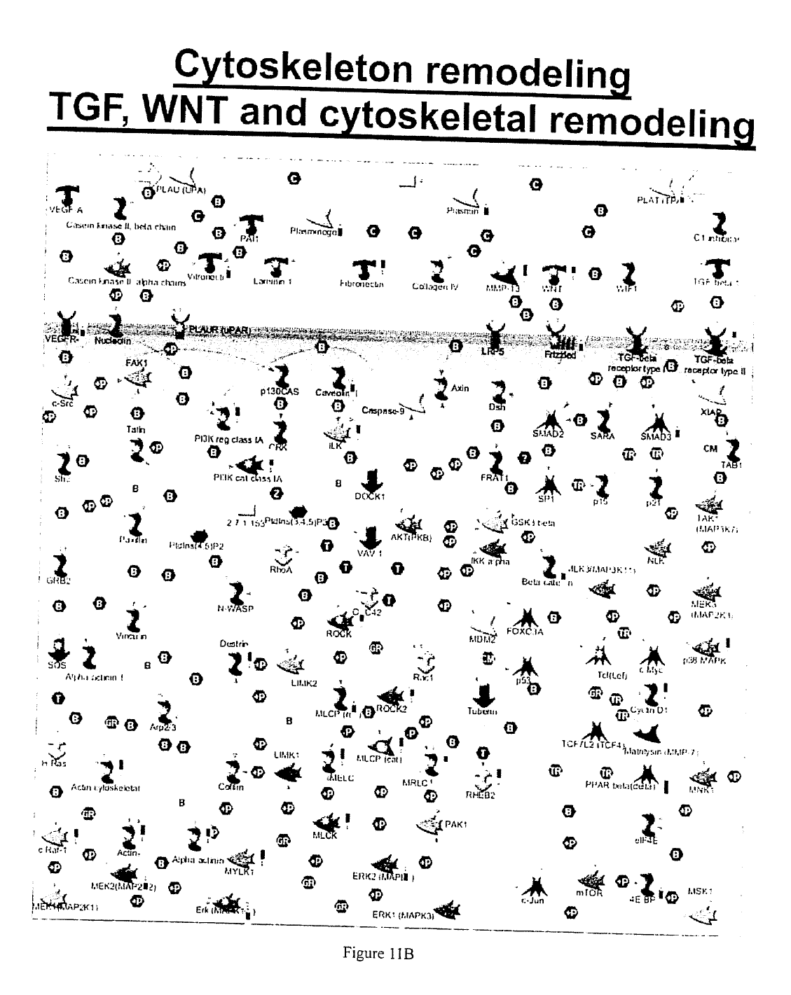

WD NW led to similar weight gain as seen in WD SW-fed mice by week 52 (Table 2). WD NW also led to an elevation of AST and ALT relative to both CD NW and administration of CD SW alone. The AST and ALT levels following WD NW were higher than seen with WD SW administration, but this did not reach significance. More importantly, WD NW-fed mice developed severe steatosis but did not have hepatocyte ballooning by week 8 and only mild ballooning by week 52 (FIG. 3B and FIG. 10A-B). They did however have some lobular inflammation at 8 weeks which increased modestly by week 52. They also developed fibrosis over time (FIG. 10). In contrast, mice fed CD SW alone did not develop significant steatosis, lobular inflammation or fibrosis. At week 8, occasional ballooning was noted but this was not seen at week 52.

Activation of Signaling Pathways Relevant for Humans with NASH:

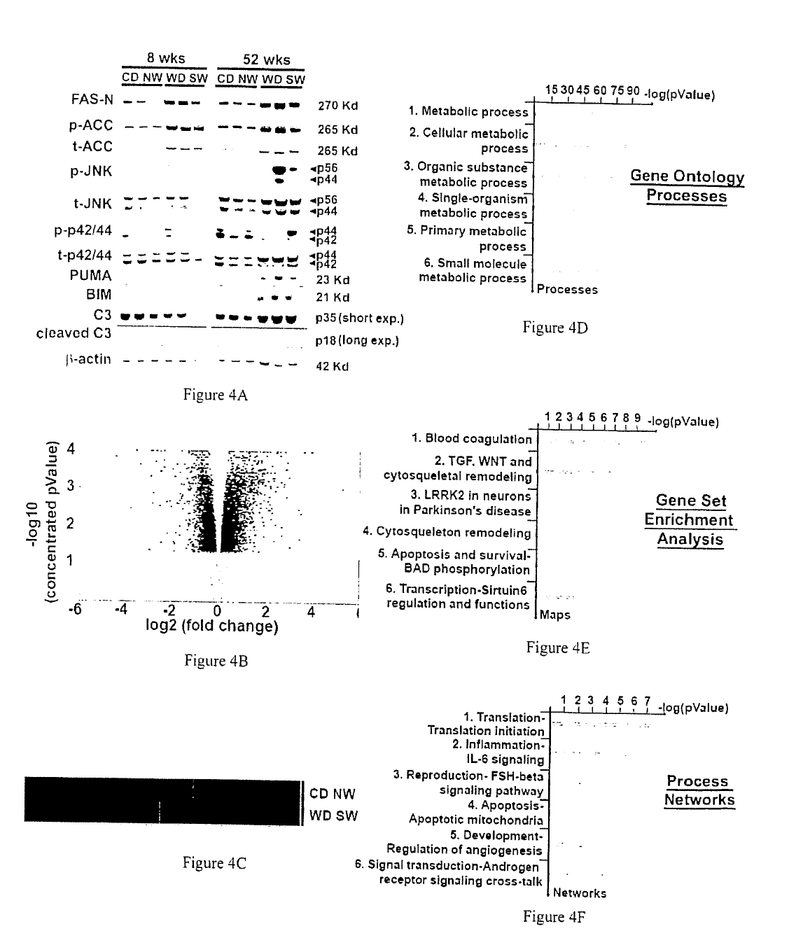

Increased lipogenic-, inflammatory- and pro-apoptotic signaling are hallmarks of NASH in humans¹⁸⁻²⁰. The activation of such signaling pathways was assessed at both early (8 week) and late (52 week) time points in CD NW- and WD SW-fed mice. At both weeks 8 and 52, there was a significant increase in expression of fatty acid synthetase (FAS) and acyl CoA carboxylase (ACC) as well as phosphorylation of acyl CoA carboxylase indicative of its activation (FIG. 4A). On the other hand, inflammatory signaling via phosphorylation of JNK, a key mediator of inflammation in human NASH^{19,21}, was not increased at week 8 but significantly increased at later time points up to week 52. There was also increased PARP and cleaved caspase 3 at week 52 indicating activation of apoptotic signaling. There was also a significant increase in BIM and PUMA proteins expression, two important pro-apoptotic signals that are also activated in human NASH and by lipotoxicity^{22,23}. These signaling pathways were activated in both male and female mice (data not shown).

WD SW-Induced NASH has a Transcriptomic Profile Similar to Humans with NASH:

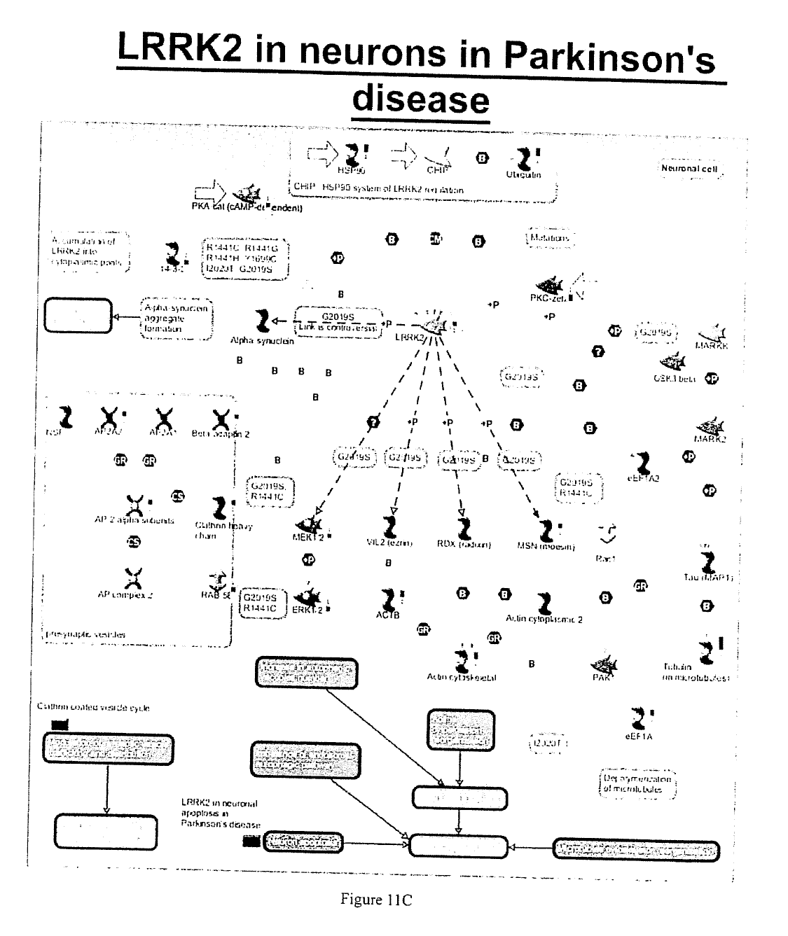

The Illumina Expression BeadChip (Illumina) platform was used to interrogate the hepatic transcriptome at early (8 week) and late (52 week) stages of NAFLD in mice fed WD SW. Volcano plot and heatmap visualization demonstrated distinct differences between CD-fed and WD SW-fed mouse liver transcriptome at 8 weeks (FIGS. 4B and 4C). The separation of gene expression profile was further confirmed by principal component analysis. There was also a remarkable similarity in gene signatures from one mouse to the other within each experimental group. The differential gene expression was related to the GENE ONTOLOGY™ database to evaluate the biological and cellular processes altered by WD SW. These demonstrated a major impact on genes involved in multiple metabolic processes at 8 weeks (FIG. 4D). Alternately, using Gene Set Enrichment Analysis (GSEA) to identify differences in a priori defined set of genes along specific pathways, several additional pathways were identified. Based on statistical significance, the top rank ordered pathways included blood coagulation, TGF-Wnt, cytoskeletal remodeling, and LRRK2 in Parkinson's disease (FIG. 4E and FIG. 11A-C). There was also evidence for increased apoptotic and inflammatory signaling pathway activation at a transcriptional level. Biological networks activated by WD SW was further determined (FIG. 4F) in an unbiased manner using the Analyze Networks algorithm^{24,25}. A β-catenin, JAK2, SMAD3, TGF-β receptor type II, ILK network that impacts cell proliferation, regulation of phosphorylation and macromolecule metabolic processes was the principal network upregulated by WD SW at 8 weeks (Table 3) (p<9.82e-14, z-score 11.7, g-score 37.9).

TABLE 3
Activated biological networks in mice fed a high fructose/sucrose,
high fat Western diet (WD SW) for 8 weeks.

| No | Network name | Processes | Size | Target | Pathways | p-Value | zScore | gScore |
|---|---|---|---|---|---|---|---|---|
| 1 | Beta-catenin, | regulation of cell | 51 | 19 | 21 | 9.82e−14 | 11.73 | 37.98 |
| | JAK2, | proliferation (78.4%), | | | | | | |
| | SMAD3, | positive regulation of | | | | | | |
| | TGF-beta | cell proliferation | | | | | | |
| | receptor type | (64.7%), regulation of | | | | | | |
| | II, ILK | phosphorylation | | | | | | |
| | | (66.7%), response to | | | | | | |
| | | organic substance | | | | | | |
| | | (84.3%), positive | | | | | | |
| | | regulation of | | | | | | |
| | | macromolecule | | | | | | |
| | | metabolic process | | | | | | |
| | | (78.4%) | | | | | | |
| 2 | MyD88, | energy coupled proton | 50 | 25 | 9 | 4.91e−22 | 16.48 | 27.73 |
| | CaMK IV, | transport, down | | | | | | |
| | IRAK1/2, | electrochemical | | | | | | |
| | p38alpha | gradient (17.8%), ATP | | | | | | |
| | (MAPK14). | synthesis coupled | | | | | | |
| | TRAF6 | proton transport | | | | | | |
| | | (17.8%), | | | | | | |
| | | mitochondrial ATP | | | | | | |
| | | synthesis coupled | | | | | | |
| | | proton transport | | | | | | |
| | | (15.6%), positive | | | | | | |
| | | regulation of protein | | | | | | |
| | | metabolic process | | | | | | |
| | | (46.7%), phosphorus | | | | | | |
| | | metabolic process | | | | | | |
| | | (62.2%) | | | | | | |
| 3 | SURF1, | hydrogen ion | 50 | 36 | 0 | 1.74e−41 | 25.27 | 25.27 |
| | CLCN3, | transmembrane | | | | | | |
| | eIF3S7, | transport (23.4%), | | | | | | |
| | C6orf62, | proton transport | | | | | | |
| | RPL9 | (23.4%), hydrogen | | | | | | |
| | | transport (23.4%), | | | | | | |
| | | monovalent inorganic | | | | | | |
| | | cation transport | | | | | | |
| | | (23.4%), positive | | | | | | |
| | | regulation of plasma | | | | | | |
| | | membrane long-chain | | | | | | |
| | | fatty acid transport | | | | | | |
| | | (6.4%) | | | | | | |

JAK2, Janus kinase 2; SMAD3, SMAD family member 3; TGF, Transforming growth factor; ILK, integrin-linked kinase; MyD88, Myeloid differentiation primary response gene 88; CaMK IV, Calmodulin-dependent protein kinase IV; IRAK1/2, IL-1 receptor-associated kinase 1/2; MAPK4, Mitogen-activated protein kinase 4; TRAF6, TNF (Tumor necrosis tumor)-receptor-associated factor 6; SURF1, Surfeit 1; CLCN3, Chloride channel, voltage sensitive 3; eIF3S7, Eukaryotic translation initiation factor 3 subunit 7; C6orf62, chromosome 6 open reading frame 62; RPL9, Ribosomal protein L9.

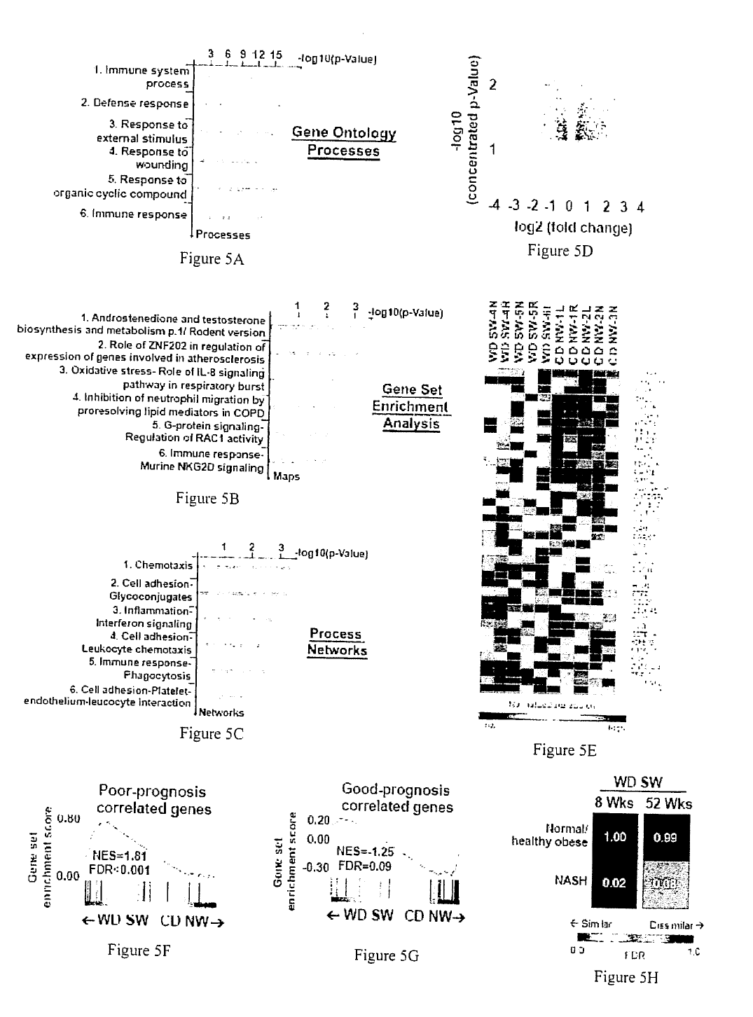

Hepatic gene expression profiles were also analyzed in mice fed a WD SW at 52 weeks and further compared to a known human gene expression databank of liver cirrhosis and NASH patients^{26,27} (FIG. 5). The hepatic gene expression at week 52 was different from both CD NW fed mice for the same duration (FIG. 5A-E) and also from mice fed WD SW for 8 weeks.

GSEA at 52 weeks identified changes in androstenedione metabolism and transcriptional regulation of lipoprotein metabolism, oxidative stress-related signaling pathways related to activation of the innate immune system and inflammatory pathways as the principal pathways that were altered (FIG. 5B) consistent with data on lipoprotein metabolism and the role of innate immunity in disease progression in humans with NASH^{26,27}. Gene ontology (GO) and process networks analysis also identified activation of multiple inflammatory processes (FIG. 5A-C). While the metabolic pathway changes noted at 8 weeks were still altered, they were less than that noted at the earlier time-point. When compared to human cirrhosis of varied etiologies including NASH (186-gene signature, including 73 poor prognosis-correlated and 113 good prognosis-correlated genes)^{28 29}, the mouse transcriptome at 52 weeks demonstrated significant concordance with cirrhosis of poor prognosis (normalized enrichment score, NES 1.81, FDR<0.001) (FIG. 5F-G). Furthermore, when compared to a human normal and NASH gene signature dataset (18 human NASH patients and 41 normal/healthy obese individuals)³⁰, there was a strong concordance of the mouse gene expression data with the NASH signature (FDR 0.02 at 8 weeks and 0.08 at 52 weeks) (FIG. 5H).

### Development of Murine HCC Resembling Human HCC:

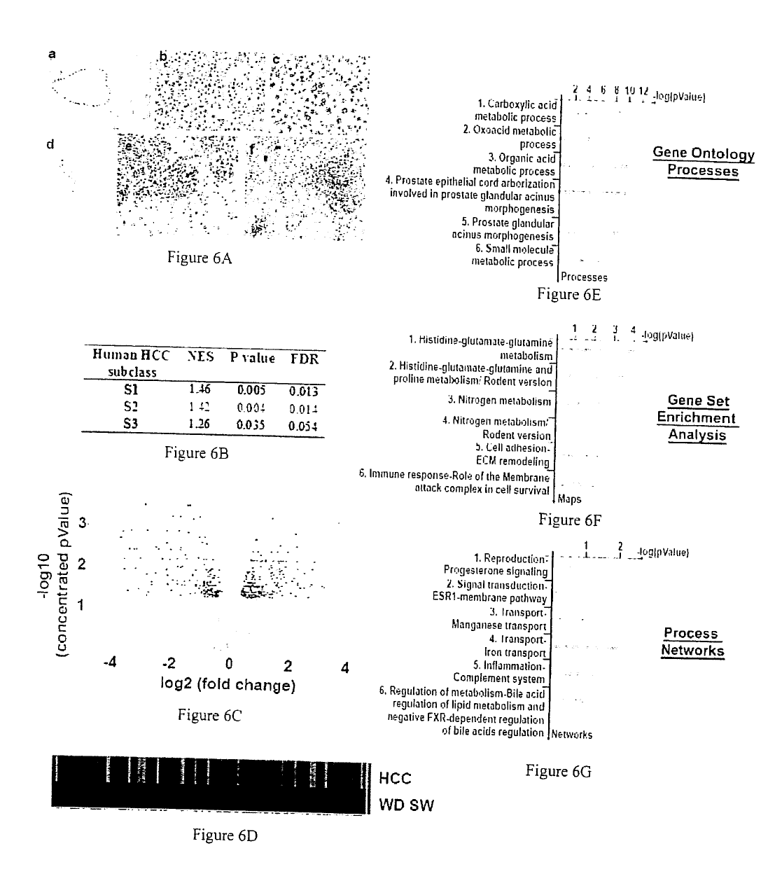

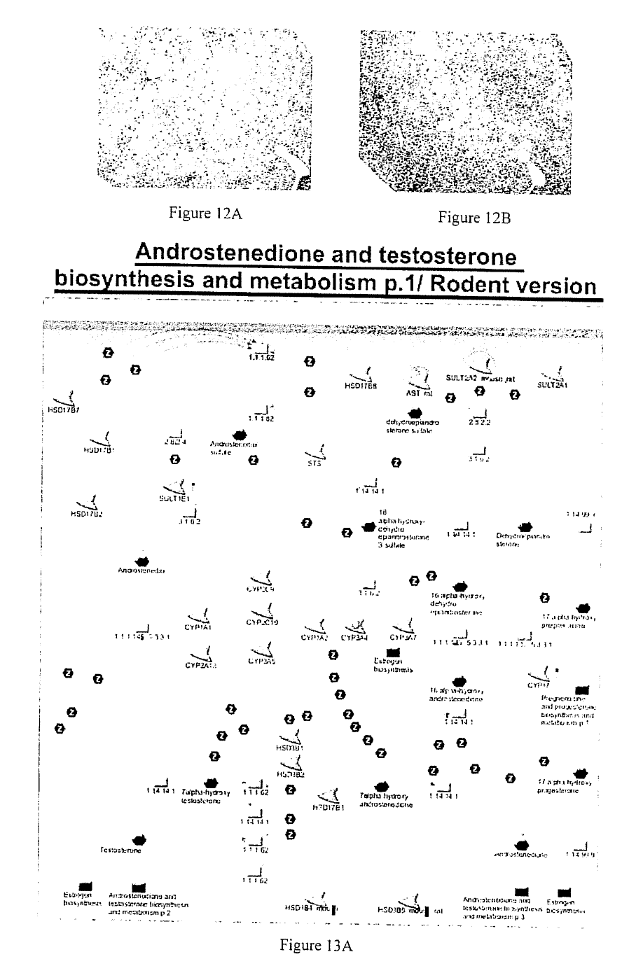

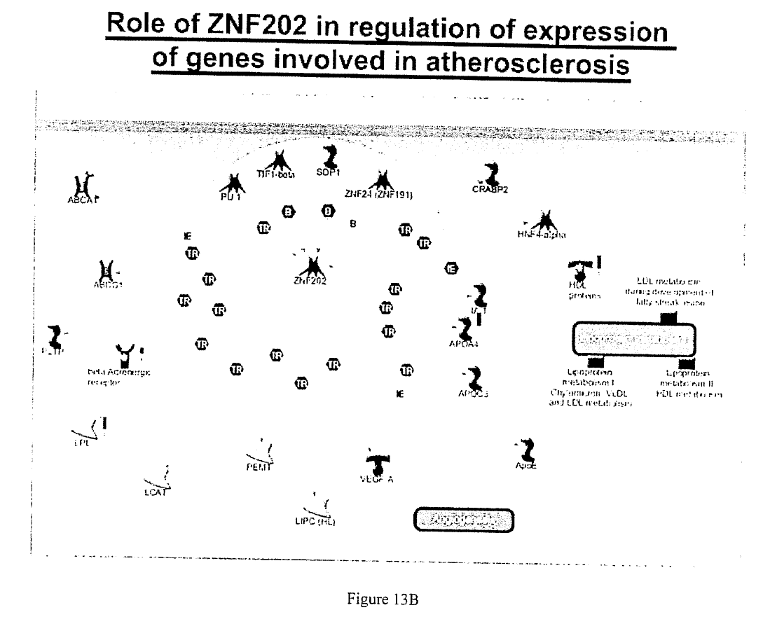

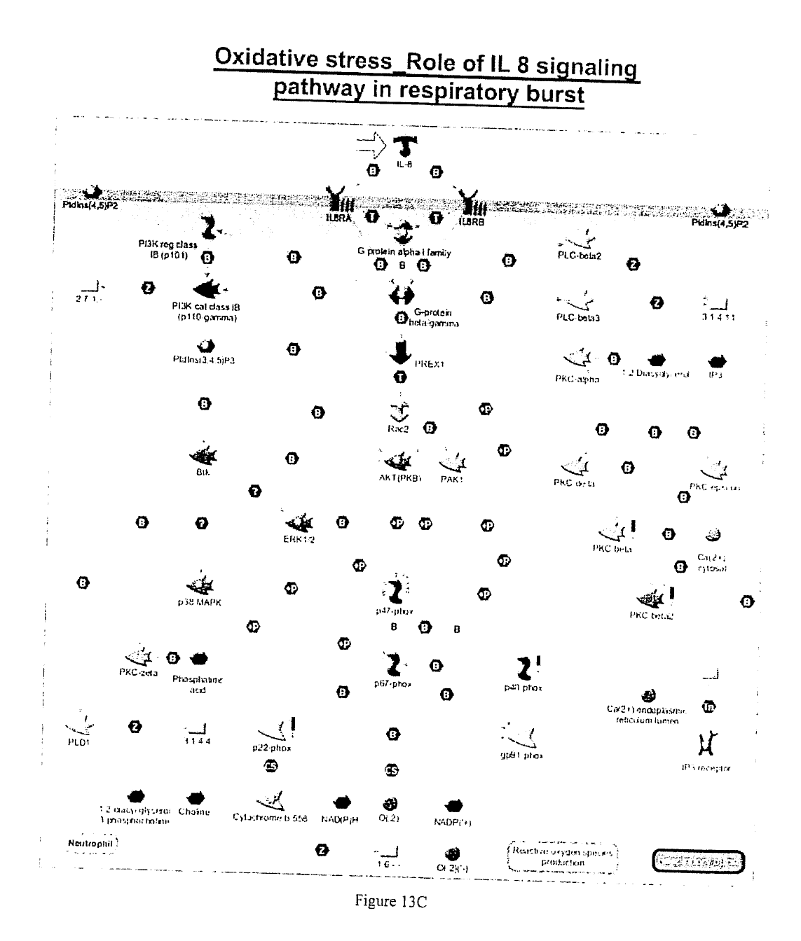

HCC developed in 90% of male mice and 75% of female mice between weeks 32-52. There were 5 or more foci of tumors in each male mouse whereas 1-2 foci were seen in females (FIGS. 2A and 6A). The tumors in males tended to be larger and some were associated with hemorrhage within the tumor. All mice had foci of well-differentiated HCC and 40% had poorly differentiated HCC (Table 4). Also, hepatic adenomas (FIG. 12A-B), some with foci of HCC within them, were noted in 25% of the mice. At a transcriptomic level, the HCC transcriptome was related to the S1 or S2 subclasses of human HCC 28 (FIG. 6B) (NES and FDR: 1.46 and 0.01, and 1.42 and 0.01 respectively). Compared to surrounding non-tumorous liver in mice on WD SW, the HCC transcriptome demonstrated activation of several pathways related to nitrogen and amino acid metabolism, oxidative stress signaling, inflammation and cell adhesion-extracellular matrix remodeling (FIG. 6C-G). Functional process network analysis (FIG. 6G and FIG. 13A-C) revealed changes related to progesterone signaling, bile acid regulation of lipid metabolism, hypoxia and oxidative stress, signal transduction-ESR1 and modulation of apoptosis induced by external signals by estrogen.

TABLE 4
Tumor rate and characteristics.

| | Predominant | | | | |
|---|---|---|---|---|---|
| | type of | Degree of | Number of | Tumor | |
| Diet (52 wks) | Steatosis | Disease | Tumors/Liver | Rate (%) | Tumor Type |
| CD NW | None | Normal | None | 0% (0/15) | None |
| CD SW | None | Normal | None | 0% (0/4) | None |
| WD NW | Microvesicular | NAFLD | 1-2 | 40% (6/15) | WD HCC |
| WD SW | Macrovesicular | NASH | ≥3 | 89% (8/9) | WD HCC (8/8) |
| | | | | | PD HCC (3/8) |
| | | | | | Adenoma (2/8) |

Mice were fed for 52 weeks either a chow diet (CD NW), a high fructose/glucose diet (CD SW), a high fat Western diet (WD NW) or a high fructose/glucose, high fat Western diet (WD SW).
Values are mean ± SEM for 4-15 mice per group;
WD HCC, Well-Differentiated Hepatocarcinoma;
PD HCC, Poorly-Differentiated Hepatocarcinoma.

There is a substantial need for models of NASH to accelerate therapeutic and biomarker development. The invention described herein provides a mouse model where fatty liver disease is induced by caloric excess as it occurs in most humans with NASH. The mice sequentially develop steatosis, steatohepatitis, progressive fibrosis and HCC. This is accompanied by weight gain, insulin resistance, hypertriglyceridemia and increased circulating levels of LDL-cholesterol. The hepatic transcriptome of these mice was also similar to that seen in human NASH from week 8 through 52. In later stages of the disease, the hepatic transcriptome from affected mice demonstrated concordance with a 186-gene signature of cirrhosis of varied etiology including NASH containing 73 genes associated with poor prognosis²⁹. Furthermore, striking similarity of global liver transcriptome between WD SW-fed mice and human NASH was observed for 8 weeks and 52 weeks. Pathways related to lipogenesis, inflammation and apoptosis that are considered to be relevant for humans with NASH^{15, 19} were also activated. These mice developed progressive fibrosis with early cirrhotic remodeling and also HCC which resembled S1 or S2 human HCC²⁸. Interestingly, the induction was relatively stronger for S1 subclass which is associated with steatohepatitic HCC variant³¹. The disease also developed over a 52 week time-frame which is analogous to about 25-30 years in a human, the time course of disease progression to cirrhosis and HCC in many chronic liver diseases^{32,33}. Together, this demonstrates that this mouse model overcomes several deficiencies of other models and mimics the key aspects of human NASH. It is therefore used as a preclinical model for this disease.

The current model impacts three important areas of liver disease for which there is an unmet need for more therapeutic development. These include NASH, hepatic fibrosis and HCC. In addition to mimicking human NASH, this model demonstrates progressive fibrosis with marked perisinusoidal fibrosis, the distinctive pattern of fibrosis in human NASH, and in some cases, bridging fibrosis and even early cirrhotic changes. Most prior approaches for anti-fibrotic drug development have utilized carbon tetrachloride, thioacetamide administration, or bile duct ligation as models of hepatic fibrosis for preclinical testing of potential therapies. These models are not representative of the human disease state and thus are not ideal for testing anti-fibrotic therapies. In the current model, fibrosis develops as a consequence of a disease process that resembles human disease and is better suited for the preclinical assessment of drugs targeting hepatic fibrosis to prevent disease progression or induce regression of advanced fibrosis. Given the high incidence of HCC in this model, it also has utility for testing preventive and therapeutic approaches against HCC.

It is interesting to note the development of hepatic adenomas as well as HCC within hepatic adenomas in our model. There are case reports of HCC arising in a hepatic adenoma in subjects with metabolic syndrome without cirrhosis³⁴. Therefore, the DIAMOND™ model also recapitulates this phenomenon and provides further opportunities to dissect the molecular basis for this observation.

It is important to note that a high fat diet alone can induce the phenotype but with lesser severity. However, sugar water administration alone did not have any impact on liver histology. This could be related to the amount of sugar consumed in this protocol which allowed ad lib consumption of sugar water. Also, female mice developed a less pronounced phenotype and a lower incidence of HCC. These gender-based differences should also permit studies that may shed novel insights on why HCC affects males with NASH more than females^{35,36}. The model is also relatively specific since mice with a pure background did not develop the phenotype.

In summary, this diet-induced animal model of nonalcoholic fatty liver disease recapitulates the various phenotypes of the disease and their associated metabolic and underlying molecular characteristics. It serves as a relevant model to identify therapeutic targets, model disease progression and test preventive and therapeutic approaches against NASH, hepatic fibrosis and HCC.

The detailed descriptions and photographs, graphs, figures, and genetic data are supportive and descriptive of the embodiments of the invention described herein, and while some of the preferred embodiments of this invention have been described in detail, various alternative designs and embodiments exist for practicing the methods disclosed herein. It is expressly noted that the present invention is not limited to the embodiments described in detail herein; rather, modifications and additions to what has been expressly described herein are also included within the scope of the invention. Moreover, it will be understood that the features of the various embodiments described herein are not generally mutually exclusive and can exist in various combinations and permutations without departing from the spirit and the scope of the invention.

### REFERENCES

Throughout this application, various references describe the state of the art to which this invention pertains. The disclosures of these references are hereby incorporated by reference into the present disclosure.
1 Bedogni, G. et al. Prevalence of and risk factors for nonalcoholic fatty liver disease: the Dionysos nutrition and liver study. Hepatology 42, 44-52, doi:10.1002/hep.20734 (2005).
2 Angulo, P. Nonalcoholic fatty liver disease. The New England journal of medicine 346, 1221-1231, doi:10.1056/NEJMra011775 (2002).
3 Charlton, M. R. et al. Frequency and outcomes of liver transplantation for nonalcoholic steatohepatitis in the United States. Gastroenterology 141, 1249-1253, doi:10.1053/j.gastro.2011.06.061 (2011).
4 Dyson, J. et al. Hepatocellular cancer: the impact of obesity, type 2 diabetes and a multidisciplinary team. Journal of hepatology 60, 110-117, doi:10.1016/j.jhep.2013.08.011 (2014).
5 Sanyal, A., Poklepovic, A., Moyneur, E. & Barghout, V. Population-based risk factors and resource utilization for HCC: US perspective. Current medical research and opinion 26, 2183-2191, doi:10.1185/03007995.2010.506375 (2010).
   6 White, D. L., Kanwal, F. & El-Serag, H. B. Association between nonalcoholic fatty liver disease and risk for hepatocellular cancer, based on systematic review. Clinical gastroenterology and hepatology: the official clinical practice journal of the American Gastroenterological Association 10, 1342-1359 e1342, doi:10.1016/j.cgh.2012.10.001 (2012).
7 Rinella, M. E. & Green, R. M. The methionine-choline deficient dietary model of steatohepatitis does not exhibit insulin resistance. Journal of hepatology 40, 47-51 (2004).
8 Yang, S., Lin, H. Z., Hwang, J., Chacko, V. P. & Diehl, A. M. Hepatic hyperplasia in noncirrhotic fatty livers: is obesity-related hepatic steatosis a premalignant condition? Cancer research 61, 5016-5023 (2001).
9 Sato, W. et al. Hepatic gene expression in hepatocyte-specific Pten deficient mice showing steatohepatitis without ethanol challenge. Hepatology research: the official journal of the Japan Society of Hepatology 34, 256-265, doi:10. 1016/j.hepres.2006.01.003 (2006).
10 Fujii, M. et al. A murine model for non-alcoholic steatohepatitis showing evidence of association between diabetes and hepatocellular carcinoma. Medical molecular morphology 46, 141-152, dooi: 10.1007/s00795-013-0016-1 (2013).
11 Ishikawa, H. et al. L-carnitine prevents progression of non-alcoholic steatohepatitis in a mouse model with upregulation of mitochondrial pathway. PloS one 9, e100627, doi:10.1371/journal.pone.0100627 (2014).
12 Dowman, J. K. et al. Development of hepatocellular carcinoma in a murine model of nonalcoholic steatohepatitis induced by use of a high-fat/fructose diet and sedentary lifestyle. The American journal of pathology 184, 1550-1561, doi:10.1016/j.ajpath.2014.01.034 (2014).
13 Lee, L. et al. Nutritional model of steatohepatitis and metabolic syndrome in the Ossabaw miniature swine. Hepatology 50, 56-67, doi:10.1002/hep.22904 (2009).
14 Newell, P., Villanueva, A., Friedman, S. L., Koike, K. & Llovet, J. M. Experimental models of hepatocellular carcinoma. Journal of hepatology 48, 858-879, doi:10.1016/j.jhep.2008.01.008 (2008).
15 Siddiqui, M. S. et al. Severity of Nonalcoholic Fatty Liver Disease and Progression to Cirrhosis Associate With Atherogenic Lipoprotein Profile. Clinical gastroenterology and hepatology: the official clinical practice journal of the American Gastroenterological Association, doi:10.1016/j.cgh.2014.10.008 (2014).
16 Ayala, J. E. et al. Standard operating procedures for describing and performing metabolic tests of glucose homeostasis in mice. Disease models & mechanisms 3, 525-534, doi:10.1242/dmm.006239 (2010).
17 Kleiner, D. E. et al. Design and validation of a histological scoring system for nonalcoholic fatty liver disease. Hepatology 41, 1313-1321, doi:10.1002/hep.20701 (2005).
18 Donnelly, K. L. et al. Sources of fatty acids stored in liver and secreted via lipoproteins in patients with nonalcoholic fatty liver disease. The Journal of clinical investigation 115, 1343-1351, doi:10.1172/JCI23621 (2005).
19 Puri, P. et al. Activation and dysregulation of the unfolded protein response in nonalcoholic fatty liver disease. Gastroenterology 134, 568-576, doi:10.1053/j.gastro.2007.10.039 (2008).
20 Feldstein, A. E. et al. Hepatocyte apoptosis and fas expression are prominent features of human nonalcoholic steatohepatitis. Gastroenterology 125, 437-443 (2003).
21 Farrell, G. C., van Rooyen, D., Gan, L. & Chitturi, S. NASH is an Inflammatory Disorder: Pathogenic, Prognostic and Therapeutic Implications. Gut and liver 6, 149-171, doi:10.5009/gn1.2012.6.2.149 (2012).
22 Barreyro, F. J. et al. Transcriptional regulation of Bim by FoxO3A mediates hepatocyte lipoapoptosis. The Journal of biological chemistry 282, 27141-27154, doi:10.1074/jbc.M704391200 (2007).
23 Cazanave, S. C. et al. JNK1-dependent PUMA expression contributes to hepatocyte lipoapoptosis. The Journal of biological chemistry 284, 26591-26602, doi:10.1074/jbc.M109.022491 (2009).
24 Ekins, S. et al. Algorithms for network analysis in systems-ADME/Tox using the MetaCore and MetaDrug platforms. Xenobiotica; the fate of foreign compounds in biological systems 36, 877-901, doi:10.1080/00498250600861660 (2006).
25 Liberzon, A. et al. Molecular signatures database (MSigDB) 3.0. Bioinformatics 27, 1739-1740, doi:10.1093/bioinformatics/btr260 (2011).
26 Henao-Mejia, J. et al. Inflammasome-mediated dysbiosis regulates progression of NAFLD and obesity. Nature 482, 179-185, doi:10.1038/nature10809 (2012).
27 Maher, J. J., Leon, P. & Ryan, J. C. Beyond insulin resistance: Innate immunity in nonalcoholic steatohepatitis. Hepatology 48, 670-678, doi:10.1002/hep.22399 (2008).
28 Hoshida, Y. et al. Integrative transcriptome analysis reveals common molecular subclasses of human hepatocellular carcinoma. Cancer research 69, 7385-7392, doi:10.1158/0008-5472.CAN-09-1089 (2009).
29 Hoshida, Y. et al. Prognostic gene expression signature for patients with hepatitis C-related early-stage cirrhosis. Gastroenterology 144, 1024-1030, doi:10.1053/j.gastro.2013.01.021 (2013).
30 Ahrens, M. et al. DNA methylation analysis in nonalcoholic fatty liver disease suggests distinct disease-specific and remodeling signatures after bariatric surgery. Cell metabolism 18, 296-302, doi:10.1016/j.cmet.2013.07.004 (2013).
31 Salomao, M., Yu, W. M., Brown, R. S., Jr., Emond, J. C. & Lefkowitch, J. H. Steatohepatitic hepatocellular carcinoma (SH-HCC): a distinctive histological variant of HCC in hepatitis C virus-related cirrhosis with associated NAFLD/NASH. The American journal of surgical pathology 34, 1630-1636, doi:10.1097/PAS.0b013e3181f31caa (2010).
32 Schwartz, M., Roayaie, S. & Konstadoulakis, M. Strategies for the management of hepatocellular carcinoma. Nature clinical practice. Oncology 4, 424-432, doi:10.1038/ncponc0844 (2007).
33 Zaltron, S., Spinetti, A., Biasi, L., Baiguera, C. & Castelli, F. Chronic HCV infection: epidemiological and clinical relevance. BMC infectious diseases 12 Suppl 2, S2, doi:10.1186/1471-2334-12-S2-S2 (2012).
34 Paradis, V. et al. Hepatocellular carcinomas in patients with metabolic syndrome often develop without significant liver fibrosis: a pathological analysis. Hepatology 49, 851-859, doi:10.1002/hep.22734 (2009).
35 Kikuchi, L. et al. Hepatocellular Carcinoma Management in Nonalcoholic Fatty Liver Disease Patients: Applicability of the BCLC Staging System. American journal of clinical oncology, doi:10.1097/COC.0000000000000134 (2014).
36 Pan, J. J. & Fallon, M. B. Gender and racial differences in nonalcoholic fatty liver disease. World journal of hepatology 6, 274-283, doi:10.4254/wjh.v6.i5.274 (2014).
37 Jung, U. J. & Choi, M. S. Obesity and its metabolic complications: the role of adipokines and the relationship between obesity, inflammation, insulin resistance, dyslipidemia and nonalcoholic fatty liver disease. International journal of molecular sciences 15, 6184-6223, doi:10.3390/ijms15046184 (2014).
38 Cordain, L. et al. Origins and evolution of the Western diet: health implications for the 21st century. The American journal of clinical nutrition 81, 341-354 (2005).
39 Charlton, M. et al. Fast food diet mouse: novel small animal model of NASH with ballooning, progressive fibrosis, and high physiological fidelity to the human condition. Am J Physiol Gastrointest Liver Physiol 301, G825-834, doi:10.1152/ajpgi.00145.2011 (2011).
40 Hafkenscheid, J. C. & Dijt, C. C. Determination of serum aminotransferases: activation by pyridoxal-5′-phosphate in relation to substrate concentration. Clinical chemistry 25, 55-59 (1979).
41 Fossati, P. & Prencipe, L. Serum triglycerides determined colorimetrically with an enzyme that produces hydrogen peroxide. Clinical chemistry 28, 2077-2080 (1982).
42 Pesce, M. A. & Bodourian, S. H. Enzymatic rate method for measuring cholesterol in serum. Clinical chemistry 22, 2042-2045 (1976).
43 Charuruks, N. & Milintagas, A. Evaluation of calculated low-density lipoprotein against a direct assay. Journal of the Medical Association of Thailand=Chotmaihet thangphaet 88 Suppl 4, S274-279 (2005).
44 Lattouf, R. et al. Picrosirius red staining: a useful tool to appraise collagen networks in normal and pathological tissues. The journal of histochemistry and cytochemistry: official journal of the Histochemistry Society 62, 751-758, doi:10.1369/0022155414545787 (2014).
45 Mehlem, A., Hagberg, C. E., Muhl, L., Eriksson, U. & Falkevall, A. Imaging of neutral lipids by oil red 0 for analyzing the metabolic status in health and disease. Nature protocols 8, 1149-1154, doi:10.1038/nprot.2013.055 (2013).
46 Contos, M. J. & Sanyal, A. J. The clinicopathologic spectrum and management of nonalcoholic fatty liver disease. Advances in anatomic pathology 9, 37-51 (2002).
47 Ludwig, J., Viggiano, T. R., McGill, D. B. & Oh, B. J. Nonalcoholic steatohepatitis: Mayo Clinic experiences with a hitherto unnamed disease. Mayo Clinic proceedings 55, 434-438 (1980).
48 Bedossa, P. & Consortium, F. P. Utility and appropriateness of the fatty liver inhibition of progression (FLIP) algorithm and steatosis, activity, and fibrosis (SAF) score in the evaluation of biopsies of nonalcoholic fatty liver disease. Hepatology 60, 565-575, doi:10.1002/hep.27173 (2014).
49 Goodman, Z. D., Becker, R. L., Jr., Pockros, P. J. & Afdhal, N. H. Progression of fibrosis in advanced chronic hepatitis C: evaluation by morphometric image analysis. Hepatology 45, 886-894, doi:10.1002/hep.21595 (2007).
50 Cheung, O. et al. Nonalcoholic steatohepatitis is associated with altered hepatic MicroRNA expression. Hepatology 48, 1810-1820, doi:10.1002/hep.22569 (2008).
51 Dillies, M. A. et al. A comprehensive evaluation of normalization methods for Illumina high-throughput RNA sequencing data analysis. Briefings in bioinformatics 14, 671-683, doi:10.1093/bib/bbs046 (2013).
52 Hoshida, Y. et al. Gene expression in fixed tissues and outcome in hepatocellular carcinoma. The New England journal of medicine 359, 1995-2004, doi:10.1056/NEJMoa0804525 (2008).
53 Hoshida, Y., Brunet, J. P., Tamayo, P., Golub, T. R. & Mesirov, J. P. Subclass mapping: identifying common subtypes in independent disease data sets. PloS one 2, e1195, doi:10.1371/journal.pone.0001195 (2007).

SEQUENCE LISTING
The patent application contains a lengthy “Sequence Listing” section. A copy of the “Sequence Listing” is available in electronic form from the USPTO web site https://bulkdata.uspto.gov/data2/lengthysequencelisting/2018/. An electronic copy of the “Sequence Listing” will also be available from the USPTO upon request and payment of the fee set forth in 37 CFR 1.19(b)(3).

1. A viable and fertile mouse whose genome comprises any or all of the chromosome sequences represented by SEQ ID NOs:1-39.

2. The mouse of claim 1, wherein administration of a high-fat and high-sugar diet causes development of one or more disease conditions selected from the group consisting of steatosis, liver fibrosis, steatohepatitis, cirrhosis, hepatocellular carcinoma, obesity, insulin resistance, and dyslipidemia.

3. A viable and fertile mouse whose genome comprises the genomic sequence obtainable from the cell culture accessible under ATCC deposit number PTA-123551 deposited on Oct. 11, 2016.

4. A method of inducing development of hepatic steatosis, progressive hepatic fibrosis, steatohepatitis, hepatocellular carcinoma, or at least one condition associated with metabolic syndrome in a mouse whose genome comprises any or all of the chromosome sequences represented by SEQ ID NOs:1-39 comprising the steps of:
a. administering to said mouse a diet comprising standard mouse chow and water for at least the first 8 weeks after birth,
b. discontinuing administration of the diet comprising standard mouse chow and water when the mouse is at least 8 weeks old, and
c. further administering to the at least 8 week old mouse a diet comprising high-fat mouse chow and sugar water for a duration of at least 4 weeks.

5. The method of claim 4, wherein the diet administered in step (c) is administered for at least 4 weeks and hepatic steatosis is induced.

6. The method of claim 4, wherein the diet administered in step (c) is administered for at least 16 weeks and progressive hepatic fibrosis is induced.

7. The method of claim 4, wherein the diet administered in step (c) is administered for at least 12 weeks and steatohepatitis is induced.

8. The method of claim 4, wherein the diet administered in step (c) is administered for at least 32 weeks and hepatocellular carcinoma is induced.

9. The method of claim 4, wherein the diet administered in step (c) is administered for at least 4 weeks and at least one disorder associated with metabolic syndrome is induced.

10. The method of claim 4, wherein the disorder associated with metabolic syndrome is selected from the group consisting of: impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, cardiovascular disease, atherosclerosis, microvascular disease, and kidney disease.

11. A method for screening a compound for the prevention or treatment of a disorder selected from the group consisting of hepatic steatosis, progressive hepatic fibrosis, steatohepatitis, hepatocellular carcinoma, and a disorder associated with metabolic syndrome in a mouse whose genome comprises any or all of the chromosome sequences represented by SEQ ID NOs:1-39 comprising the steps of:
a. administering to said mouse a diet comprising standard mouse chow diet and water for at least the first 8 weeks after birth,
b. discontinuing administration of standard mouse chow diet and water in step (a) when the mouse is at least 8 weeks old,
c. further administering to the at least 8 week old mouse a diet comprising high-fat mouse chow and sugar water for a duration of at least 4 weeks,
d. administering said compound concurrent with step (c) or after step (c), and
e. determining the effect of the compound on said disorder relative to a mouse not treated with the compound.

12. The method of claim 11, wherein said disorder is hepatic steatosis and the diet administered in step (c) is administered for at least 4 weeks.

13. The method of claim 11, wherein said disorder is progressive hepatic fibrosis and the diet administered in step (c) is administered for at least 16 weeks.

14. The method of claim 11, wherein said disorder is steatohepatitis and the diet administered in step (c) is administered for at least 12 weeks.

15. The method of claim 11, wherein said disorder is hepatocellular carcinoma and the diet administered in step (c) is administered for at least 32 weeks.

16. The method of claim 11, wherein said disorder is a disorder associated with metabolic syndrome and the diet administered in step (c) is administered for at least 4 weeks.

17. The method of claim 11, wherein the disorder associated with metabolic syndrome is selected from the group consisting of: impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, cardiovascular disease, atherosclerosis, microvascular disease, and kidney disease.

18. An in vivo model system for at least one condition selected from the group consisting of hepatic steatosis, progressive hepatic fibrosis, steatohepatitis, hepatocellular carcinoma, and a condition associated with metabolic syndrome comprising
a viable and fertile mouse whose genome comprises any or all of the chromosome sequences represented by SEQ ID NOs:1-39, wherein said at least one condition is induced by
a. administering to said mouse a diet comprising standard mouse chow and water for at least the first 8 weeks after birth,
b. discontinuing administration of the diet comprising standard mouse chow and water when the mouse is at least 8 weeks old, and
c. further administering to the at least 8 week old mouse a diet comprising high-fat mouse chow and sugar water for a duration of at least 4 weeks.

19. The method of claim 18, wherein the diet administered in step (c) is administered for at least 4 weeks and hepatic steatosis is induced.

20. The method of claim 18, wherein the diet administered in step (c) is administered for at least 16 weeks and progressive hepatic fibrosis is induced.

21. The method of claim 18, wherein the diet administered in step (c) is administered for at least 12 weeks and steatohepatitis is induced.

22. The method of claim 18, wherein the diet administered in step (c) is administered for at least 32 weeks and hepatocellular carcinoma is induced.

23. The method of claim 18, wherein the diet administered in step (c) is administered for at least 4 weeks and at least one disorder associated with metabolic syndrome is induced.

24. The method of claim 18, wherein the disorder associated with metabolic syndrome is selected from the group consisting of: impaired glucose tolerance, whole body insulin resistance, hepatic insulin resistance, muscular insulin resistance, adipose tissue insulin resistance, incretin abnormalities, hyper-insulinemia, impaired glucose disposal rate, obesity, dyslipidemia, cardiovascular disease, atherosclerosis, microvascular disease, and kidney disease.

25. Use of an in vivo model system according to claim 18 for:
a. the study of at least one condition selected from the group consisting of hepatic steatosis, progressive hepatic fibrosis, steatohepatitis, hepatocellular carcinoma, and a condition associated with metabolic syndrome, or
b. in vivo screening or testing of the efficacy of candidate drugs for the treatment of a condition recited in a).
